(19) United States
(12) Patent Application Publication
(10) Pub. No.: US 2002/0081590 A1
(43) Pub. Date: Jun. 27, 2002

(54) METHODS AND APPARATUS FOR PREDICTING, CONFIRMING, AND DISPLAYING FUNCTIONAL INFORMATION DERIVED FROM GENOMIC SEQUENCE

(75) Inventors: Dr. Sharron G. Penn, San Mateo, CA (US); Dr. David R. Rank, Fremont, CA (US); Dr. David K. Hanzel, Palo Alto, CA (US)

Correspondence Address: Daniel M. Becker, James F. Haley, Jr., Fish & Neave, 525 University Avenue, Palo Alto, CA, 94301, US, dbecker@fishneave.com, (650) 617-4058, (650) 566-4122

(73) Assignee: AEOMICA, INC., Sunnyvale, 94086-4520 (US)

(21) Appl. No.: 09/774,203

(22) Filed: Jan. 29, 2001

Related U.S. Application Data
(63) Continuation-in-part of application No. 09/632,366, filed on Aug. 3, 2000.
(63) Continuation-in-part of application No. 09/608,408, filed on Jun. 30, 2000.
(60) Provisional application No. 60/236,359, filed on Sep. 27, 2000.
(60) Provisional application No. 60/234,687, filed on Sep. 21, 2000.
(60) Provisional application No. 60/207,456, filed on May 26, 2000.
(60) Provisional application No. 60/180,312, filed on Feb. 4, 2000.

(30) Foreign Application Priority Data
Oct. 4, 2000 (GB) 0024263.6

(51) Int. Cl. C12Q 1/68; G06F 19/00; G01N 33/48; G01N 33/50

(52) U.S. Cl. 435/6; 702/20

(57) ABSTRACT

### Abstract

Methods and apparatus for predicting, confirming and displaying functional regions from genomic sequence data are presented. The methods and apparatus are particularly useful for predicting coding regions within genomic sequence data, confirming the expression thereof experimentally, and relating and displaying the expression data in meaningful relationship to the genomic sequence. The methods and apparatus of the present invention thus present powerful tools for novel gene discovery.

### Cross Reference To Related Applications

The present application claims priority to and incorporates by reference in their entireties:

### Background of the Invention

For almost two decades following the invention of general techniques for nucleic acid sequencing, Sanger et al., Proc. Natl. Acad. Sci. USA 70(4):1209-13 (1973); Gilbert et al., Proc. Natl. Acad. Sci. USA 70(12):3581-4 (1973), these techniques were used principally as tools to further the understanding of proteins — known or suspected — about which a basic foundation of biologic knowledge had already been built. In many cases, the cloning effort that preceded sequence identification had been both informed and directed by that antecedent biological understanding.

For example, the cloning of the T cell receptor for antigen was predicated upon its known or suspected cell type-specific expression, by its suspected membrane association, and by the predicted assembly of its gene via T cell-specific somatic recombination. Hedrick et al., Nature 308(5955):149-53 (1984). Subsequent sequencing efforts at once confirmed and extended understanding of this family of proteins. Hedrick et al., Nature 308(5955):153-8 (1984).

More recently, however, the development of high throughput sequencing methods and devices, in concert with large public and private undertakings to sequence the human and other genomes, has altered this investigational paradigm: today, sequence information often precedes understanding of the basic biology of the encoded protein product.

One of the approaches to large-scale sequencing is predicated upon the proposition that expressed sequences — that is, those accessible through isolation of mRNA — are of greatest initial interest. This "expressed sequence tag" ("EST") approach has already yielded vast amounts of sequence data. Adams et al., Science 252:1651 (1991); Williamson, Drug Discov. Today 4:115 (1999); Strausberg et al., Nature Genet. 15:415 (1997); Adams et al., Nature 377(suppl.):3 (1995); Marra et al., Nature Genet. 21:191 (1999). For nucleic acids sequenced by this approach, often the only biologic information that is known a priori with any certainty is the likelihood of biologic expression itself. By virtue of the species and tissue from which the mRNA had originally been obtained, most such sequences are also annotated with the identity of the species and at least one tissue in which expression appears likely.

More recently, the pace of genomic sequencing has accelerated dramatically. When genomic DNA serves as the initial substrate for sequencing efforts, expression cannot be presumed; often the only a priori biologic information about the sequence includes the species and chromosome (and perhaps chromosomal map location) of origin.

With the ever-accelerating pace of sequence accumulation by directed, EST, and genomic sequencing approaches — and in particular, with the accumulation of sequence information from multiple genera, from multiple species within genera, and from multiple individuals within a species— there is an increasing need for methods that rapidly and effectively permit the functions of nucleic sequences to be elucidated. And as such functional information accumulates, there is a further need for methods of storing such functional information in meaningful and useful relationship to the sequence itself; that is, there is an increasing need for means and apparatus for annotating raw sequence data with known or predicted functional information.

Although the increase in the pace of genomic sequencing is due in large part to technological changes in sequencing strategies and instrumentation, Service, Science 280:995 (1998); Pennisi, Science 283: 1822-1823 (1999), there is an important functional motivation as well.

While it was understood that the EST approach would rarely be able to yield sequence information about the noncoding portions of the genome, it now also appears the EST approach is capable of capturing only a fraction of a genome's actual expression complexity.

For example, when the C. elegans genome was fully sequenced, gene prediction algorithms identified over 19,000 potential genes, of which only 7,000 had been found by EST sequencing. C. elegans Sequencing Consortium, Science 282:2012 (1998). Analogously, the recently completed sequence of chromosome 2 of Arabidopsis predicts over 4000 genes, Lin et al., Nature, 402:761 (1999), of which only about 6% had previously been identified via EST sequencing efforts. Although the human genome has the greatest depth of EST coverage, it is still woefully short of surrendering all of its genes. One recent estimate suggests that the human genome contains more than 146,000 genes, which would at this point leave greater than half of the genes undiscovered. It is now predicted that many genes, perhaps 20 to 50%, will only be found by genomic sequencing.

There is, therefore, a need for methods that permit the functional regions of genomic sequence— and most importantly, but not exclusively, regions that function to encode genes— to be identified.

Much of the coding sequence of the human genome is not homologous to known genes, making detection of open reading frames ("ORFs") and predictions of gene function difficult. Computational methods exist for predicting coding regions in eukaryotic genomes. Gene prediction programs such as GRAIL and GRAIL II, Uberbacher et al., Proc. Natl. Acad. Sci. USA 88(24):11261-5 (1991); Xu et al., Genet. Eng. 16:241-53 (1994); Uberbacher et al., Methods Enzymol. 266:259-81 (1996); GENEFINDER, Solovyev et al., Nucl. Acids. Res. 22:5156-63 (1994); Solovyev et al., Ismb 5:294-302 (1997); and GENSCAN, Burge et al., J. Mol. Biol. 268:78-94 (1997), predict many putative genes without known homology or function. Such programs are known, however, to give high false positive rates. Burset et al., Genomics 34:353-367 (1996). Using a consensus obtained by a plurality of such programs is known to increase the reliability of calling exons from genomic sequence. Ansari-Lari et al., Genome Res. 8(1):29-40 (1998).

Identification of functional genes from genomic data remains, however, an imperfect art. For example, in reporting the full sequence of human chromosome 21, the Chromosome 21 Mapping and Sequencing Consortium reports that prior bioinformatic estimates of human gene number may need to be revised substantially downwards. Nature 405:311-199 (2000); Reeves, Nature 405:283-284 (2000).

Thus, there is a need for methods and apparatus that permit the functions of the regions identified bioinformatically — and specifically, that permit the expression of regions predicted to encode protein — readily to be confirmed experimentally.

Recently, the development of nucleic acid microarrays has made possible the automated and highly parallel measurement of gene expression. Reviewed in Schena (ed.), DNA Microarrays : A Practical Approach (Practical Approach Series), Oxford University Press (1999) (ISBN: 0199637768); Nature Genet. 21(1)(suppl):1-60 (1999); Schena (ed.), Microarray Biochip: Tools and Technology, Eaton Publishing Company/BioTechniques Books Division (2000) (ISBN: 1881299376), the disclosures of which are incorporated herein by reference in their entireties.

It is common for microarrays to be derived from cDNA/EST libraries, either from those previously described in the literature, such as those from the I.M.A.G.E. consortium, Lennon et al., "The I.M.A.G.E. Consortium: an Integrated Molecular Analysis of Genomes and Their Expression," Genomics 33(1):151-2 (1996), or from the construction of "problem specific" libraries targeted at a particular biological question, R.S. Thomas et al., Toxicologist 54:68-69 (2000). Such microarrays by definition can measure expression only of those genes found in EST libraries, and thus have not been useful as probes for genes discovered solely by genomic sequencing.

The utility of using whole genome nucleic acid microarrays to answer certain biologic questions has been demonstrated for the yeast Saccharomyces cerevisiae. De Risi et al., Science 278:680 (1997). The vast majority of yeast nuclear genes, approximately 95% however, are single exon genes, i.e., lack introns, Lopez et al., RNA 5:1135-1137 (1999); Goffeau et al., Science 274:563-67 (1996), permitting coding regions more readily to be identified. Whole genome nucleic acid microarrays have not generally been used to probe gene expression from more complex eukaryotic genomes, and in particular from those averaging more than one intron per gene.

### Summary of the Invention

The present invention solves these and other problems in the art by providing methods and apparatus for predicting, confirming, and displaying functional information derived from genomic sequence.

In one aspect, the invention provides a process for predicting functional regions from genomic sequence, confirming and characterizing the functional activity of such regions experimentally, and then associating and displaying the information so obtained in meaningful and useful relationship to the original sequence data.

In a related aspect, the present invention provides apparatus for verifying the expression of putative genes identified within genomic sequence. In particular, the invention provides novel genome-derived single exon nucleic acid microarrays useful for verifying the expression of putative genes identified within genomic sequence.

In another aspect, the present invention provides compositions and kits for the ready production of nucleic acids identical in sequence to, or substantially identical in sequence to, probes on the genome-derived single exon microarrays of the present invention.

In further aspect, the present invention provides a genome-derived single-exon microarray packaged together with such an ordered set of amplifiable probes corresponding to the probes, or one or more subsets of probes, thereon. In alternative embodiments, the ordered set of amplifiable probes is packaged separately from the genome-derived single exon microarray.

In another aspect, the invention provides means for displaying annotated sequence, and in particular, for displaying sequence annotated according to the methods and apparatus of the present invention. Further, such display can be used as a preferred graphical user interface for electronic search, query, and analysis of such annotated sequence.

In another aspect, the invention provides genome-derived single exon nucleic acid probes useful for gene expression analysis, and particularly for gene expression analysis by microarray. The invention particularly provides genome-derived single-exon probes known to be expressed in one or more tissues.

FIELD OF THE INVENTION: The present invention is in the fields of bioinformatics and molecular biology, and relates particularly to analytical methods and apparatus for predicting, confirming, and displaying functional information derived from genomic sequence. The invention particularly relates to methods and apparatus for identifying portions of genomic sequence data that encode genes, to the design, manufacture and use of genome-derived single-exon nucleic acid microarrays for assaying expression thereof, and to methods and apparatus for display of genomic sequence annotated with expression information.

### Brief Description of the Drawings

The above and other objects and advantages of the present invention will be apparent upon consideration of the following detailed description taken in conjunction with the accompanying drawings, in which like characters refer to like parts throughout, and in which:

FIG. 1 illustrates a process for predicting functional regions from genomic sequence, confirming the functional activity of such regions experimentally, and associating and displaying the data so obtained in meaningful and useful relationship to the original sequence data, according to the present invention;

FIG. 2 further elaborates that portion of the process schematized in FIG. 1 for predicting functional regions from genomic sequence, according to the present invention;

FIG. 3 illustrates a visual display according to the present invention, herein denominated a "Mondrian", in which a single genomic sequence is annotated with predicted and experimentally confirmed functional information;

FIG. 4 presents a Mondrian of a hypothetical annotated genomic sequence, further identifying typical color conventions when the Mondrian is used to annotate genomic sequence with exon-specific expression data, as in FIGS. 9 and 10;

FIG. 5 is a chart that summarizes data from experimental Example 1, showing the size distributions of predicted exon length (dashed line) and actual PCR products (amplicons) (solid line) as obtained from human genomic sequence according to the methods of the present invention;

FIG. 6 is a histogram that summarizes data from experimental Examples 1 and 2, showing the number of tissues in which predicted exons could be shown to be expressed using simultaneous two color hybridization to a genome-derived single exon microarray of the present invention. The graph shows the number of sequence-verified products that were either not expressed in any of the ten tested tissues/cell types ("0"), expressed in one or more but not all tested tissues ("1" - "9"), or expressed in all tissues tested ("10");

FIG. 7 is a pictorial representation of data from experimental Examples 1 and 2, showing the expression (ratio relative to control) of probes having verified sequences that were expressed with signal intensity greater than 3 in at least one tissue, with: FIG. 7A showing both the expression as measured by microarray hybridization in each of the 10 measured tissues and the expression as measured "bioinformatically" by query of EST, NR and SwissProt databases; with FIG. 7B showing the legend for display of physical expression (ratio) in FIG. 7A; and with FIG. 7C showing the legend for scoring EST hits as depicted in FIG. 7A;

FIG. 8 is a chart of data from experimental Examples 1 and 2, showing a comparison of normalized CY3 signal intensity for arrayed sequences that were identical to sequences in existing EST, NR and SwissProt databases (known) or that were dissimilar (unknown), where the dashed line denotes the signal intensity for all sequence-verified products with a BLAST Expect ("E") value of greater than 1e-30 (1 x 10⁻³⁰) ("unknown") and the solid line denotes sequence-verified spots with a BLAST expect ("E") value of less than 1e-30 (1 x 10⁻³⁰)("known");

FIG. 9 presents a Mondrian of BAC AC008172 (bases 25,000 to 130,000), containing the carbamyl phosphate synthetase gene (AF154830.1); and

FIG. 10 is a Mondrian of BAC A049839.

### Detailed Description of the Invention

Definitions

As used herein, the term "microarray" and equivalent phrase "nucleic acid microarray" refer to a substrate-bound collection of plural nucleic acids, hybridization to each of the plurality of bound nucleic acids being separately detectable. The substrate can be solid or porous, planar or non-planar, unitary or distributed.

As so defined, the term "microarray" and phrase "nucleic acid microarray" include all the devices so called in Schena (ed.), DNA Microarrays: A Practical Approach (Practical Approach Series), Oxford University Press (1999) (ISBN: 0199637768); Nature Genet. 21(1)(suppl):1-60 (1999); and Schena (ed.), Microarray Biochip: Tools and Technology, Eaton Publishing Company/BioTechniques Books Division (2000) (ISBN: 1881299376), the disclosures of which are incorporated herein by reference in their entireties.

As so defined, the term "microarray" and phrase "nucleic acid microarray" also include substrate-bound collections of plural nucleic acids in which the nucleic acids are distributably disposed on a plurality of beads, rather than on a unitary planar substrate, as is described, inter alia, in Brenner et al., Proc. Natl. Acad. Sci. USA 97(4):166501670 (2000), the disclosure of which is incorporated herein by reference in its entirety; in such case, the term "microarray" and phrase "nucleic acid microarray" refer to the plurality of beads in aggregate.

As used herein with respect to a nucleic acid microarray, the term "probe" refers to the nucleic acid that is, or is intended to be, bound to the substrate. As used herein with respect to solution phase hybridization, the term "probe" refers to the nucleic acid of known sequence that is, or is intended to be, detectably labeled. In either such context, the term "target" refers to nucleic acid intended to be bound to probe by Watson-Crick complementarity.

As used herein, the expression "probe comprising SEQ ID NO", and variants thereof, intends a nucleic acid probe, at least a portion of which probe has either (i) the sequence directly as given in the referenced SEQ ID NO, or (ii) a sequence complementary to the sequence as given in the referenced SEQ ID NO, the choice as between sequence directly as given and complement thereof dictated by the requirement that the probe be complementary to the desired target.

As used herein, the phrase "expression of a probe" and its linguistic variants means that the probe hybridizes detectably at high stringency to nucleic acids that derive from mRNA.

As used herein, the term "exon" refers to a nucleic acid sequence bioinformatically predicted to encode a portion of a natural protein.

As used herein, the phrase "open reading frame" and the equivalent acronym "ORF" refer to that portion of an exon that can be translated in its entirety into a sequence of contiguous amino acids. As so defined, an ORF is wholly contained within its respective exon and has length, measured in nucleotides, exactly divisible by 3. As so defined, an ORF need not encode the entirety of a natural protein.

As used herein, the phrase "alternative splicing" and its linguistic equivalents includes all types of RNA processing that lead to expression of plural protein isoforms from a single gene; accordingly, the phrase "splice variant(s)" and its linguistic equivalents embraces mRNAs transcribed from a given gene that, however processed, collectively encode plural protein isoforms.

For example, and by way of illustration only, splice variants can include exon insertions, exon extensions, exon truncations, exon deletions, alternatives in the 5' untranslated region ("5' UT") and alternatives in the 3' untranslated region ("3' UT"). Such 3' alternatives include, for example, differences in the site of RNA transcript cleavage and site of poly(A) addition. See, e.g., Gautheret et al., Genome Res. 8:524-530 (1998).

As used herein, the phrase "specific binding pair" intends a pair of molecules that bind to one another with high specificity. Binding pairs typically have affinity or avidity of at least 10⁷, preferably at least 10⁸, more preferably at least 10⁹ liters/mole. Nonlimiting examples of specific binding pairs are: antibody and antigen; biotin and avidin; and biotin and streptavidin.

As used herein with respect to the visual display of annotated genomic sequence, the term "rectangle" means any geometric shape that has at least a first and a second border, wherein each of the first and second borders is capable of mapping uniquely to a point of another visual object of the display.

Methods and Apparatus for Identifying, Confirming, and Displaying Functional Regions of Genomic Sequence

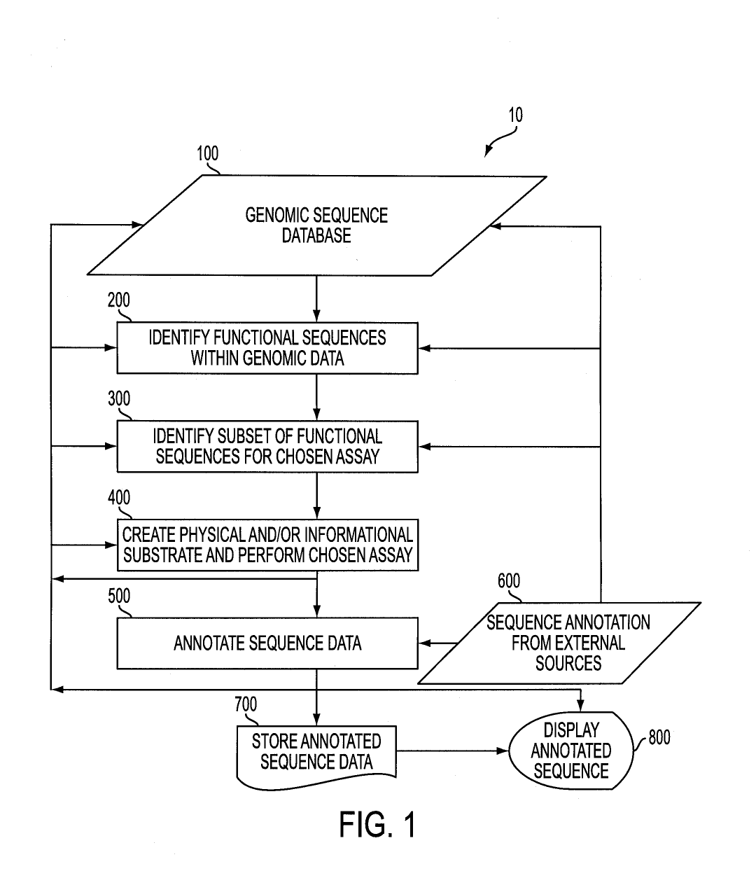

FIG. 1 is a flow chart illustrating in broad outline a first aspect of the present invention, a process for predicting functional regions from genomic sequence, confirming and characterizing the functional activity of such regions experimentally, and then associating and displaying the information so obtained in meaningful and useful relationship to the original genomic sequence data.

The initial input into process 10 of the present invention is drawn from one or more databases 100 containing genomic sequence data. Because genomic sequence is usually obtained from subgenomic fragments, the sequence data typically will be stored in a series of records corresponding to these subgenomic sequenced fragments. Some fragments will have been catenated to form larger contiguous sequences ("contigs"); others will not. A finite percentage of sequence data in the database will typically be erroneous, consisting inter alia of vector sequence, sequence created from aberrant cloning events, sequence of artificial polylinkers, and sequence that was erroneously read.

Each sequence record in database 100 will minimally contain as annotation a unique sequence identifier (accession number), and will typically be annotated further to identify the date of accession, species of origin, and depositor. Because database 100 can contain nongenomic sequence, each sequence will typically be annotated further to permit query for genomic sequence. Chromosomal origin, optionally with map location, can also be present. Data can be, and over time increasingly will be, further annotated with additional information, in part through use of the present invention, as described below. Annotation can be present within the data records, in information external to database 100 and linked to the records thereto, or through a combination of the two.

Databases useful as genomic sequence database 100 in the present invention include GenBank, and particularly include several divisions thereof, including the htgs (draft), NT (nucleotide, command line), and NR (nonredundant) divisions. GenBank is produced by the National Institutes of Health and is maintained by the National Center for Biotechnology Information (NCBI). Databases of genomic sequence from species other than human, such as mouse, rat, Arabidopsis thaliana, C. elegans, C. brigsii, Drosophilamelanogaster, zebra fish, and other higher eukaryotic organisms will also prove useful as genomic sequence database 100.

Genomic sequence obtained by query of genomic sequence database 100 is then input into one or more processes 200 for identification of regions therein that are predicted to have a biological function as specified by the user. Such functions include, but are not limited to, encoding protein, regulating transcription, regulating message transport after transcription, regulating message splicing after transcription, regulating message degradation after transcription, contributing to or controlling chromosomal somatic recombination, contributing to chromosomal stability or movement, contributing to allelic exclusion or X chromosome inactivation, and the like.

The particular genomic sequence to be input into process 200 will depend upon the function for which relevant sequence is to be identified as well as upon the approach chosen for such identification. Process step 200 can be iterated to identify different functions within a given genomic region. In such case, the input often will be different for the several iterations.

Sequences predicted to have the requisite function by process 200 are then input into process 300 where a subset of the input sequences suitable for experimental confirmation is identified. Experimental confirmation can involve physical and/or bioinformatic assay. Where the subsequent experimental assay is bioinformatic, rather than physical, there are fewer constraints on the sequences that can be tested, and in this latter case therefore process 300 can output the entirety of the input sequence.

The subset of sequences output from process 300 is then used in process 400 for experimental verification and characterization of the function predicted in process 200, which experimental verification can, and often will, include both physical and bioinformatic assay.

Process 500 annotates the sequence data with the functional information obtained in the physical and/or bioinformatic assays of process 400. Such annotation can be done using any technique that usefully relates the functional information to the sequence, as, for example, by incorporating the functional data into the sequence data record itself, by linking records in a hierarchical or relational database, by linking to external databases, by a combination thereof, or by other means well known within the database arts. The data can even be submitted for incorporation into databases maintained by others, such as GenBank, which is maintained by NCBI.

As further noted in FIG. 1, additional annotation can be input into process 500 from external sources 600.

The annotated data is then optionally displayed in process 800, either before, concomitantly with, or after optional storage 700 on nontransient media, such as magnetic disk, optical disc, magnetooptical disk, flash memory, or the like.

FIG. 1 shows that the experimental data output from process 400 can be used in each preceding step of process 10: e.g., facilitating identification of functional sequences in process 200, facilitating identification of an experimentally suitable subset thereof in process 300, and facilitating creation of physical and/or informational substrates for, and performance of subsequent assay, of functional sequences in process 400.

Information from each step can be passed directly to the succeeding process, or stored in permanent or interim form prior to passage to the succeeding process. Often, data will be stored after each, or at least a plurality, of such process steps. Any or all process steps can be automated.

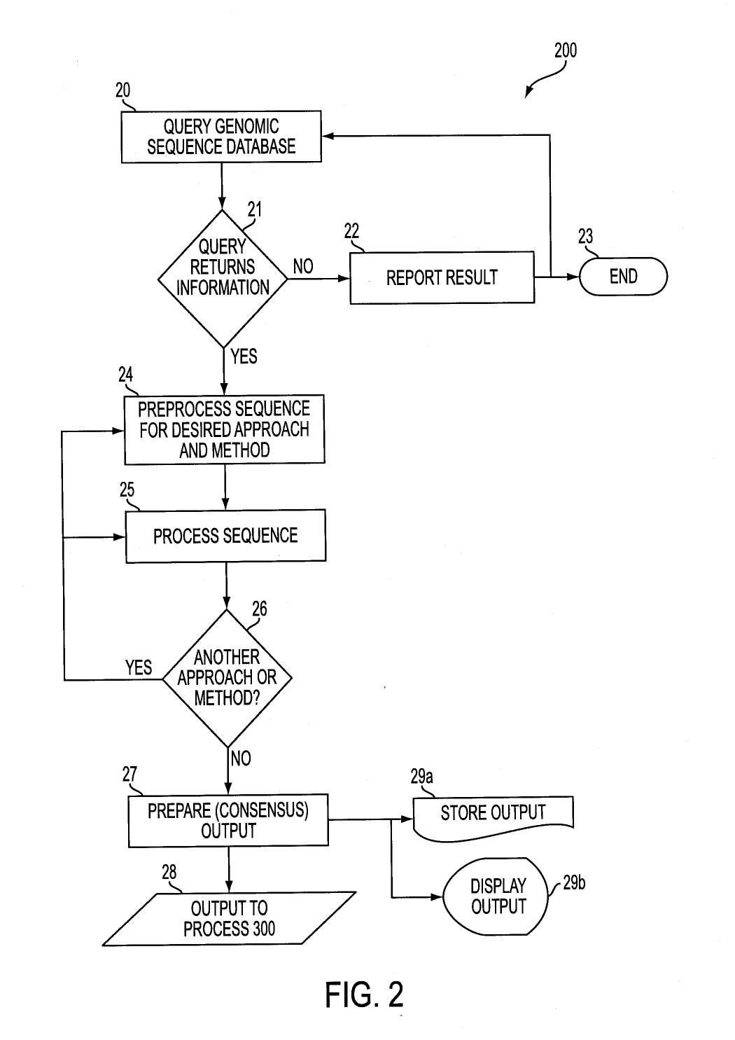

FIG. 2 further elaborates the prediction of functional sequence within genomic sequence according to process 200.

Genomic sequence database 100 is first queried 20 for genomic sequence.

The sequence required to be returned by query 20 will depend, in the first instance, upon the function to be identified.

For example, genomic sequences that function to encode protein can be identified inter alia using gene prediction approaches, comparative sequence analysis approaches, or combinations of the two. In gene prediction analysis, sequence from one genome is input into process 200 where at least one, preferably a plurality, of algorithmic methods are applied to identify putative coding regions. In comparative sequence analysis, by contrast, corresponding, e.g., syntenic, sequence from a plurality of sources, typically a plurality of species, is input into process 200, where at least one, possibly a plurality, of algorithmic methods are applied to compare the sequences and identify regions of least variability.

The exact content of query 20 will also depend upon the database queried. For example, if the database contains both genomic and nongenomic sequence, perhaps derived from multiple species, and the function to be predicted is protein coding in human genomic DNA, the query will accordingly require that the sequence returned be genomic and derived from humans.

Query 20 can also incorporate criteria that compel return of sequence that meets operative requirements of the subsequent analytical method. Alternatively, or in addition, such operative criteria can be enforced in subsequent preprocess step 24.

For example, if the function sought to be identified is protein coding, query 20 can incorporate criteria that return from genomic sequence database 100 only those sequences present within contigs sufficiently long as to have obviated substantial fragmentation of any given exon among a plurality of separate sequence fragments.

Such criteria can, for example, consist of a required minimal individual genomic sequence fragment length, such as 10 kb, more typically 20 kb, 30 kb, 40kb, and preferably 50 kb or more, as well as an optional further or alternative requirement that sequence from any given clone, such as a bacterial artificial chromosome ("BAC"), be presented in no more than a finite maximal number of fragments, such as no more than 20 separate pieces, more typically no more than 15 fragments, even more typically no more than about 10-12 fragments.

Our results have shown that genomic sequence from bacterial artificial chromosomes (BACs) is sufficient for gene prediction analysis according to the present invention if the sequence is at least 50 kb in length, and if additionally the sequence from any given BAC is presented in fewer than 15, and preferably fewer than 10, fragments. Accordingly, query 20 can incorporate a requirement that data accessioned from BAC sequencing be in fewer than 15, preferably fewer than 10, fragments.

An additional criterion that can be incorporated into the query can be the date, or range of dates, of sequence accession. Although the process has been described above as if genomic sequence database 100 were static, it is of course understood that the genomic sequence databases need not be static, and indeed are typically updated on a frequent, even hourly, basis. Thus, as further described in experimental Examples 1 and 2, infra, it is possible to query the database for newly added sequence, either newly added after an absolute date or newly added relative to a prior analysis performed using the methods and apparatus of the present invention. In this way, the process herein described can incorporate a dynamic, temporal component.

One utility of such temporal limitation is to identify, from newly accessioned genomic sequence, the presence of novel genes, particularly those not previously identified by EST sequencing (or other sequencing efforts that are similarly based upon gene expression). As further described in Example 1, such an approach has shown that newly accessioned human genomic sequence, when analyzed for sequences that function to encode protein, readily identifies genes that are novel over those in existing EST and other expression databases. In fact, as shown below, fully 2/3 of genes identified in newly accessioned human genomic sequence have not hitherto been identified. This makes the methods of the present invention extremely powerful gene discovery tools.

And as would be appreciated, such gene discovery can be performed using genomic sequence from species other than human. Particularly useful species are those used as model systems during drug development, such as rodent, particularly mouse.

If query 20 incorporates multiple criteria, such as above-described, the multiple criteria can be performed as a series of separate queries or as a single query, depending in part upon the query language, the complexity of the query, and other considerations well known in the database arts.

If query 20 returns no genomic sequence meeting the query criteria, the negative result can be reported by process 22, and process 200 (and indeed, entire process 10) ended 23, as shown. Alternatively, or in addition to report and termination of the initial inquiry, a new query 20 can be generated that takes into account the initial negative result.

When query 20 returns sequence meeting the query criteria, the returned sequence is then passed to optional preprocessing 24, suitable and specific for the desired analytical approach and the particular analytical methods thereof to be used in process 25.

Preprocessing 24 can include processes suitable for many approaches and methods thereof, as well as processes specifically suited for the intended subsequent analysis.

Preprocessing 24 suitable for most approaches and methods will include elimination of sequence irrelevant to, or that would interfere with, the subsequent analysis. Such sequence includes repetitive sequence, such as Alu repeats and LINE elements, vector sequence, artificial sequence, such as artificial polylinkers, and the like. Such removal can readily be performed by identification and subsequent masking of the undesired sequence.

Identification can be effected by comparing the genomic sequence returned by query 20 with public or private databases containing known repetitive sequence, vector sequence, artificial sequence, and other artifactual sequence. Such comparison can readily be done using programs well known in the art, such as CROSS-MATCH or REPEATMASKER, the latter available on-line at http://ftp.genome.washington.edu/RM/RepeatMasker.html, or by proprietary sequence comparison programs the engineering of which is well within the skill in the art.

Alternatively, or in addition, undesirable, including artifactual, sequence can be identified algorithmically without comparison to external databases and thereafter removed. For example, synthetic polylinker sequence can be identified by an algorithm that identifies a significantly higher than average density of known restriction sites. As another example, vector sequence can be identified by algorithms that identify nucleotide or codon usage at variance with that of the bulk of the genomic sequence.

Once identified, undesired sequence can be removed. Removal can usefully be done by masking the undesired sequence as, for example, by converting the specific nucleotide references to one that is unrecognized by the subsequent bioinformatic algorithms, such as "X". Alternatively, but at present less preferred, the undesired sequence can be excised from the returned genomic sequence, leaving gaps.

Preprocessing 24 can further include selection from among duplicative sequences of that one sequence of highest quality. Higher quality can be measured as a lower percentage of, fewest number of, or least densely clustered occurrence of ambiguous nucleotides, defined as those nucleotides that are identified in the genomic sequence using symbols indicating ambiguity. Higher quality can also or alternatively be valued by presence in the longest contig.

Preprocessing 24 can, and often will, also include formatting of the data as specifically appropriate for passage to the analytical algorithms of process 25. Such formatting can and typically will include, inter alia, addition of a unique sequence identifier, either derived from the original accession number in genomic sequence database 100, or newly applied, and can further include additional annotation. Formatting can include conversion from one to another sequence listing standard, such as conversion to or from FASTA or the like, depending upon the input expected by the subsequent process.

Preprocessing, which can be optional depending upon the function desired to be identified and the informational requirements of the methods for effecting such identification, is followed by sequence processing 25, where sequences with the desired function are identified within the genomic sequence.

As mentioned above, such functions can include, but are not limited to, encoding protein, regulating transcription, regulating message transport after transcription, regulating message splicing after transcription, regulating message degradation after transcription, contributing to or controlling chromosomal somatic recombination, contributing to chromosomal stability or movement, contributing to allelic exclusion or X chromosome inactivation, and the like.

Where the function specified is protein coding, the above-described process of the present invention can be used rapidly and efficiently to identify individual exons in genomic sequence.

As discussed below, and further described in detail in commonly owned and copending U.S. provisional application nos. 60/207,456, filed May 26, 2000; 60/234,687, filed September 21, 2000; 60/236,359, filed September 27, 2000; in commonly owned and copending U.K. patent application no. 0024263.6, filed October 4, 2000; and in commonly owned and copending PCT applications PCT/US01/00666; PCT/US01/00667; PCT/US01/00664; PCT/US01/00669; PCT/US01/00665; PCT/US01/00668; PCT/US01/00663; PCT/US01/00662; PCT/US01/00661; and PCT/US01/00670, the disclosures of which are incorporated herein by reference in their entirety, we have used the methods and apparatus of the present invention to identify more than 15,000 exons in human genomic sequence whose expression we have confirmed in at least one human tissue or cell type. Fully two-thirds of the exons belong to genes that were not at the time of our discovery represented in existing public expression (EST, cDNA) databases, making the methods and apparatus of the present invention extremely powerful tools for novel gene discovery.

And as further mentioned below and described in detail in commonly owned and copending U.S. patent application no. 09/632,366, filed August 3, 2000, the disclosure of which is incorporated herein by reference in its entirety, the genome-derived single exon probes and microarrays of the present invention prove exceedingly useful in the high throughput identification of a large variety of alternative splice events in eukaryotic cells and tissues.

To identify such individual exons from genomic sequence, process 25 is used to identify putative coding regions. Two exemplary approaches useful in process 25 for identifying sequence that encodes putative genes are gene prediction and comparative sequence analysis.

Gene prediction can be performed using any of a number of algorithmic methods, embodied in one or more software programs, that identify open reading frames (ORFs) using a variety of heuristics, such as GRAIL, DICTION, GENSCAN, and GENEFINDER.

Comparative sequence analysis similarly can be performed using any of a variety of known programs that identify regions with lower sequence variability.

An advantage of comparative sequence analysis is that genomic sequence can be input into process 200 that is less comprehensive and/or of lesser quality than that required by gene prediction programs.

We have, for example, recently used comparative sequence analysis to identify sequences that are orthologous as between human and mouse genomes, and output the mouse sequences so identified ("similons") into process 300; this has permitted us to identify, and then to identify expression of, novel mouse exons and genes. As is well known in the pharmaceutical arts, genes identified in model systems provide targets for assessing the value of targets for therapeutic intervention and screening for and assessing agents that interact with those targets.

As further described in Example 1, below, gene prediction software programs yield a range of results. For the newly accessioned human genomic sequence input in Example 1, for example, GRAIL identified the greatest percentage of genomic sequence as putative coding region, 2% of the data analyzed; GENEFINDER was second, calling 1%; and DICTION yielded the least putative coding region, with 0.8% of genomic sequence called as coding region.

Increased reliability can be obtained when consensus is required among several such methods. Although discussed herein particularly with respect to exon calling, consensus among methods will in general increase reliability of predicting other functions as well.

Thus, as indicated by query 26, sequence processing 25, optionally with preprocessing 24, can be repeated with a different method, with consensus among such iterations determined and reported in process 27.

Process 27 compares the several outputs for a given input genomic sequence and identifies consensus among the separately reported results. The consensus itself, as well as the sequence meeting that consensus, is then stored in process 29a, displayed in process 29b, and/or output to process 300 for subsequent identification of a subset thereof suitable for assay.

Multiple levels of consensus can be calculated and reported by process 27.

For example, as further described in Example 1infra, process 27 can report consensus as between all specific pairs of methods of gene prediction, as consensus among any one or more of the pairs of methods of gene prediction, or as among all of the gene prediction algorithms used. Thus, in Example 1, process 27 reported that GRAIL and GENEFINDER programs agreed on 0.7% of genomic sequence, that GRAIL and DICTION agreed on 0.5% of genomic sequence, and that the three programs together agreed on 0.25% of the data analyzed. Put another way, 0.25% of the genomic sequence was identified by all three of the programs as containing putative coding region.

As another example, three of the four gene prediction algorithms that we presently use — GENEFINDER, GENSCAN, and GRAIL— predict frame information in addition to the position of exons. If there is overlap in position and frame of the predicted exons, even if not complete identity, the predicted exons are merged in process 27 to generate the largest possible consensus coding region. The process is iterated until all possible overlaps have been merged. This approach reduces the mean number of exons present in each amplicon, and is preferred in generating exon-specific probes useful for detecting exon elongation and exon truncation alternative splice events.

Furthermore, consensus can be required among different approaches to identifying a chosen function.

For example, if the function desired to be identified is coding of protein sequence, and a first used approach to exon calling is gene prediction, the process can be repeated on the same input sequence, or subset thereof, with another approach, such as comparative sequence analysis. In such a case, where comparative sequence analysis follows gene prediction, the comparison can be performed not only on genomic nucleic acid sequence, but additionally or alternatively can be performed on the predicted amino acid sequence translated from exons prior-identified by the gene prediction approach.

Although shown as an iterative process, the multiple analyses required to achieve consensus can be done in series, in parallel, or some combination thereof.

Predicted functional sequence, optionally representing a consensus among a plurality of methods and approaches for determination thereof, is passed to process 300 for identification of a subset thereof for functional assay.

Where the function sought to be identified is protein coding, process 300 is used to identify a subset thereof suitable for experimental verification by physical and/or bioinformatic approaches.

Where the goal is the identification and confirmation of expression of only a single exon of gene — for example, to provide a gene-specific probe — exons identified in process 200 can be classified, or binned, bioinformatically into putative genes. This binning can be based inter alia upon consideration of the average number of exons/gene in the species chosen for analysis, upon density of exons that have been called on the genomic sequence, and other empirical rules; the putative gene structure is also provided by various of these gene prediction programs. Thereafter, one or more among the exons can be chosen for subsequent use in gene expression assay.

Where the goal is, instead, the identification and confirmation of expression of all, or of a plurality, of the exons of a gene — as is desired for detection of alternative splice events, as further described in commonly owned and copending U.S. patent application serial no. 09/632,366, filed August 3, 2000, the disclosure of which is incorporated herein by reference in its entirety — putative exons identified in process 200 can be classified, or binned, bioinformatically into putative genes. Thereafter, all of the exon-specific exons can be chosen for subsequent confirmation in gene expression assay.

Where such subsequent gene expression assay uses amplified nucleic acid, considerations such as desired amplicon length, primer synthesis requirements, putative exon length, sequence GC content, existence of possible secondary structure, and the like can be used to identify and select those exons that appear most likely successfully to amplify. Where subsequent gene expression assay relies upon nucleic acid hybridization, whether or not using amplified product, further considerations involving hybridization stringency can be applied to identify that subset of sequences that will most readily permit sequence-specific discrimination at a chosen hybridization and wash stringency. One particular such consideration is avoidance of putative exons that span repetitive sequence; such sequence can hybridize spuriously to nonspecific message, reducing specific signal in the hybridization.

For bioinformatic assay, there are fewer constraints on the sequences that can be tested experimentally, and in this latter case therefore process 300 can output the entirety of the input sequence.

The subset of sequences identified by process 300 as suitable for use in assay is then used in process 400 to create the physical and/or informational substrate for experimental verification of the predictions made in process 200, and thereafter to assay those substrates.

Where the goal is to identify protein coding regions in genomic sequence, the expression of the sequences predicted to encode protein is verified in process 400.

Thus, in another aspect, the present invention provides methods and apparatus for verifying the expression of putative exons identified within genomic sequence. In particular, the invention provides methods for verifying gene expression in which expression of predicted exons is measured and confirmed using a novel type of nucleic acid microarray, the genome-derived single exon nucleic acid microarray of the present invention.

According to one embodiment of this aspect, predicted exons are amplified from genomic DNA.

Amplification can be performed using the polymerase chain reaction (PCR). Although PCR is conveniently used, other amplification approaches, such as rolling circle amplification, can also be used.

Amplification schemes can be designed to capture the entirety of each predicted exon in an amplicon with minimal additional (that is, flanking intronic or intergenic) sequence. Because exons predicted from genomic sequence using the methods of the present invention differ in length, such an approach results in amplicons of varying length.

However, we have found that most exons predicted from human genomic sequence are shorter than 500 bp in length. Although amplicons of at least about 75 base pairs, more preferably at least about 100 base pairs, even more preferably at least about 200 base pairs can be immobilized as probes on nucleic acid microarrays, our early experimental results using the methods of the present invention suggested that longer amplicons, at least about 400 base pairs, more preferably about 500 base pairs, are more effectively immobilized on glass slides or other prepared surfaces.

Although we had suspected that the intronic and intergenic material flanking putative exons in such longer amplicons might cause interference with exon-specific hybridization during microarray experiments, we have found instead, to our surprise, that the ratio of expression of any such probe as between an experimental tissue (or cell type) and a control tissue is not significantly affected by the presence in the probes of sequence that does not contribute to hybridization to message or cDNA.

Equally surprising, the art had suggested that single exon probes would not provide sufficient signal intensity for high stringency hybridization analyses. Although low stringency hybridization conditions have been designed that permit informative hybridization to highly redundant oligonucleotide-based microarrays, it was believed that the high stringency hybridization conditions typically used for EST-based microarrays would not be usable with single exon probes. We have found, surprisingly, that single-exon probes provide adequate signal at high stringency.

As a result, we have found that we are readily able to use genome-derived amplification products having a single exon flanked by intergenic and/or intronic sequence to confirm the expression of bioinformatically predicted exons.

To the extent that chemical synthesis methods permit oligonucleotides to be generated of sufficient length to encompass an exon, such oligonucleotides can be used as probes in lieu of amplified material. At present, however, amplified products can be generated that exceed the reasonable size limit of chemically synthesized oligonucleotides; amplification thus more readily permits probes to be generated that have single exons flanked by intronic and/or intergenic sequence.

Probes having flanking intergenic and/or intronic sequence permit a wider range of alternative splice events to be detected than do probes that contain only exonic sequence. For example, exon extension would be detectable with such probes as an increase in signal intensity: we have found a near-linear relationship between signal intensity and length of hybridizing sequence. And when used to assay heteronuclear, i.e., immature mRNA, probes having intronic and/or intergenic flanking sequence permit a wider variety of events to be assessed.

Furthermore, certain advantages derive from application to the microarray of amplicons of defined size.

Therefore, amplification schemes can alternatively, and preferably, be designed to amplify regions of defined size, preferably at least about 300 bp, more preferably at least about 400 bp, most preferably about 500 bp, centered about each predicted exon. Such an approach results in a population of amplicons of limited size diversity, but that typically contain intronic and/or intergenic nucleic acid in addition to, and flanking, the putative exon.

Conversely, somewhat fewer than 10% of exons predicted from human genomic sequence according to the methods of the present invention exceed 500 bp in length. Portions of such longer exons, preferably at least about 300 bp, more preferably at least about 400 bp, most preferably about 500 bp, can be amplified. However, in our early experiments we found that the percentage success at amplifying pieces of such exons is low, and that such putative exons are more effectively amplified when larger fragments, at least about 1000 bp, typically at least about 1500 bp, and even as large as 2000 bp are amplified. Further routine optimization of the PCR reaction would permit 500 bp portions of the longer exons to be amplified.

For amplification, the putative exons selected in process 300 are input into one or more primer design programs, such as PRIMER3 (available online for use at http://www-genome.wi.mit.edu/cgi-bin/primer/ ), with a goal of amplifying at least about 500 base pairs of genomic sequence centered within or about exons predicted to be no more than about 500 bp, or at least about 1000 - 1500 bp of genomic sequence for exons predicted to exceed 500 bp in length, and the primers synthesized by standard techniques. Primers with the requisite sequences can be purchased commercially or synthesized by standard techniques.

Conveniently, a first predetermined sequence can be added commonly to each exon-specific 5' primer and a second, typically different, predetermined sequence commonly added to each 3' exon-unique primer. This serves to immortalize the amplicon: that is, it serves to permit further amplification of any amplicon using a single set of primers complementary respectively to the common 5' and common 3' sequence elements. The presence of these "universal" priming sequences further facilitates later sequence verification, providing a sequence common to all amplicons at which to prime sequencing reactions. The common 5' and 3' sequences can further serve to add a cloning site should any of the exons warrant further study.

Such predetermined sequence is usefully at least about 10 nt in length, typically at least about 12 nt, more typically about 15 nt in length, and usually does not exceed about 25 nt in length. The "universal" priming sequences used in the examples presented infra were each 16 nt long, and are further described in commonly owned and copending U.S. patent application serial no. 09/608,408, filed June 30, 2000, the disclosure of which is incorporated herein by reference in its entirety.

The genomic DNA to be used as substrate for amplification will come from the eukaryotic species from which the genomic sequence data had originally been obtained, or a closely related species, and can conveniently be prepared by well known techniques from somatic or germline tissue or cultured cells of the organism. See, e.g., Short Protocols in Molecular Biology : A Compendium of Methods from Current Protocols in Molecular Biology, Ausubel et al. (eds.), 4^{th} edition (April 1999), John Wiley & Sons (ISBN: 047132938X) and Maniatis et al., Molecular Cloning : A Laboratory Manual, 2^{nd} edition (December 1989), Cold Spring Harbor Laboratory Press (ISBN: 0879693096), the disclosures of which are incorporated herein by reference in their entireties. Many such prepared genomic DNAs are available commercially, with the human genomic DNAs additionally having certification of donor informed consent.

After partial purification, as by size exclusion spin column or adsorption to glass, with or without confirmation as to amplicon quality as by gel electrophoresis, each amplicon (single exon probe) is disposed in an array upon a support substrate.

Methods for creating microarrays by deposition and fixation of nucleic acids onto support substrates are well known in the art. Reviewed in Schena (ed.), DNA Microarrays : A Practical Approach (Practical Approach Series), Oxford University Press (1999) (ISBN: 0199637768); Nature Genet. 21(1)(suppl):1-60 (1999); Schena (ed.), Microarray Biochip: Tools and Technology, Eaton Publishing Company/BioTechniques Books Division (2000) (ISBN: 1881299376), the disclosures of which are incorporated herein by reference in their entireties.

Typically, the support substrate can be glass, although other materials, such as amorphous silicon, crystalline silicon, or plastics, can be used. Such plastics include polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, or mixtures thereof. Typically, the support can be rectangular, although other shapes, particularly circular disks and even spheres, present certain advantages. Particularly advantageous alternatives to glass slides as support substrates for array of nucleic acids are optical discs, as described in Demers, "Spatially Addressable Combinatorial Chemical Arrays in CD-ROM Format," international patent publication WO 98/12559, incorporated herein by reference in its entirety.

The amplified nucleic acids can be attached covalently to a surface of the support substrate or, more typically, applied to a derivatized surface in a chaotropic agent that facilitates denaturation and adherence by presumed noncovalent interactions, or some combination thereof.

Robotic spotting devices useful for arraying nucleic acids on support substrates can be constructed using public domain specifications (The MGuide, version 2.0, http://cmgm.stanford.edu/pbrown/mguide/index.html), or can conveniently be purchased from commercial sources (MicroArray GenII Spotter and MicroArray GenIII Spotter, Molecular Dynamics, Inc., Sunnyvale, CA). Spotting can also be effected by printing methods, including those using ink jet technology.

As is well known in the art, microarrays typically also contain immobilized control nucleic acids. For controls useful in providing measurements of background signal for the genome-derived single exon microarrays of the present invention, a plurality of E. coli genes can readily be used. As further described in Example 1, 16 or 32 E. coli genes suffice to provide a robust measure of nonspecific hybridization in such microarrays.

As is well known in the art, the amplified product disposed in arrays on a support substrate to create a nucleic acid microarray can consist entirely of natural nucleotides linked by phosphodiester bonds, or alternatively can include either nonnative nucleotides, alternative internucleotide linkages, or both, so long as complementary binding can be obtained in the hybridization reaction. If enzymatic amplification is used to produce the immobilized probes, the amplifying enzyme will impose certain further constraints upon the types of nucleic acid analogs that can be generated.

Although particularly described herein as using high density microarrays constructed on planar substrates, the methods of the present invention for confirming the expression of exons predicted from genomic sequence can use any of the known types of microarrays as herein defined, including microarrays on nonplanar, nonunitary, distributed substrates, such as the nonplanar, bead-based microarrays as are described in Brenner et al., Proc. Natl. Acad. Sci. USA 97(4):166501670 (2000); U.S. Patent No. 6,057,107; and U.S. Patent No. 5,736,330, the disclosures of which are incorporated herein by reference in their entireties. In theory, a packed collection of such beads provides in aggregate a higher density of nucleic acid probe than can be achieved with spotting or lithography techniques on a single planar substrate.

In addition, gene expression can be confirmed using hybridization to lower density arrays, such as those constructed on membranes, such as nitrocellulose, nylon, and positively-charged derivatized nylon membranes.

Planar microarrays on solid substrates, however, provide certain useful advantages, including compatibility with existing readers. For example, each standard microscope slide can include at least 1000, typically at least 2000, preferably 5000 or more, and up to 19,000 or more nucleic acid probes of discrete sequence.

Each putative gene can be represented in the array by a single predicted exon or by a plurality of exons predicted to belong to the same gene. And as is well known in the art, each probe of defined sequence, representing a single predicted exon, can be deposited in a plurality of locations on a single microarray to provide redundancy of signal.

The genome-derived single exon microarrays described above are an important aspect of the present invention, and differ in several fundamental and advantageous ways from microarrays presently used in the gene expression art, including (1) those created by deposition of mRNA-derived nucleic acids, (2) those created by in situ synthesis of oligonucleotide probes, and (3) those constructed from yeast genomic DNA.

Most nucleic acid microarrays that are in use for study of eukaryotic gene expression have as immobilized probes nucleic acids that are derived — either directly or indirectly — from expressed message. It is common, for example, for such microarrays to be derived from cDNA/EST libraries, either from those previously described in the literature, such as those from the I.M.A.G.E. consortium, Lennon et al., "The I.M.A.G.E. Consortium: an Integrated Molecular Analysis of Genomes and Their Expression," Genomics 33(1):151-2 (1996), or from the de novo construction of "problem specific" libraries targeted at a particular biological question, R.S. Thomas et al., Toxicologist 54:68-69 (2000), incorporated herein by reference in their entireties. Such microarrays are herein collectively denominated "EST microarrays".

Such EST microarrays by definition can measure expression only of those genes found in EST libraries, which we show herein (see infra) to represent only a fraction of expressed genes. Thus, as further discussed in Example 1, infra, fully 2/3 of genes identified from newly-accessioned human genomic sequence data by the methods of the present invention — which expression was subsequently confirmed using the methods and apparatus of the present invention — do not appear in EST or other expression databases, and could not, therefore, have been represented as probes on an EST microarray.

Furthermore, EST and cDNA libraries — and thus microarrays based thereupon — are biased by the tissue or cell type of message origin.

In addition, representation of a message in an EST and/or cDNA library depends upon the successful reverse transcription, optionally but typically with subsequent successful cloning, of the message. This introduces substantial bias into the population of probes available for arraying in EST microarrays. For example, as we show in the examples, infra, the subset of genes identified from genomic sequence by the methods of the present invention that had previously been accessioned in EST or other expression databases is biased toward genes with higher expression levels.

In contrast, neither reverse transcription nor cloning is required to produce the probes arrayed on the genome-derived single exon microarrays of the present invention. And although the ultimate deposition of a probe on the genome-derived single exon microarray of the present invention depends upon a successful amplification from genomic material, a priori knowledge of the sequence of the desired amplicon affords greater opportunity to recover any given probe sequence recalcitrant to amplification than is afforded by the requirement for successful reverse transcription and cloning of unknown message in EST approaches. Furthermore, if the sequence cannot be amplified, the sequence can at times be chemically synthesized in its entirety for use in the present invention.

Thus, the genome-derived single exon microarrays of the present invention present a far greater diversity of probes for measuring gene expression, with far less bias, than do EST microarrays presently used in the art.

As a further consequence of their ultimate origin from expressed message, the probes in EST microarrays often contain poly-A (or complementary poly-T) stretches derived from the poly-A tail of mature mRNA. These homopolymeric stretches contribute to cross-hybridization, that is, to a spurious signal occasioned by hybridization to the homopolymeric tail of a labeled cDNA that lacks sequence homology to the gene-specific portion of the probe.

In contrast, the probes arrayed in the genome-derived single exon microarrays of the present invention lack homopolymeric stretches derived from message polyadenylation, and thus can provide more specific signal. Typically, at least about 50% of the probes on the genome-derived single exon microarrays of the present invention lack homopolymeric regions consisting of A or T, where a homopolymeric region is defined for purposes herein as stretches of 25 or more, typically 30 or more, identical nucleotides. More typically, at least about 60%, even more typically at least about 75%, of probes on the genome-derived single exon microarrays of the present invention lack such homopolymeric stretches.

A further distinction, which also affects the specificity of hybridization, is occasioned by the typical derivation of EST microarray probes from cloned material. Because much of the probe material disposed as probes on EST microarrays is excised or amplified from plasmid, phage, or phagemid vectors, EST microarrays typically include a fair amount of vector sequence, more so when the probes are amplified, rather than excised, from the vector.

In contrast, the vast majority of probes in the genome-derived single exon microarrays of the present invention contain no prokaryotic or bacteriophage vector sequence, having been amplified directly or indirectly from genomic DNA. Typically, therefore, at least about 50%, more typically at least about 60%, 70%, and even 80% or more of individual exon-including probes disposed on a genome-derived single exon microarray of the present invention lack vector sequence, and particularly lack sequences drawn from plasmids and bacteriophage. Preferably, at least about 85%, more preferably at least about 90%, most preferably more than 90% of exon-including probes in the genome-derived single exon microarray of the present invention lack vector sequence. With attention to removal of vector sequences through preprocessing 24, percentages of vector-free exon-including probes can be as high as 99%. The substantial absence of vector sequence from the genome-derived single exon microarrays of the present invention results in greater specificity during hybridization, since spurious cross-hybridization to a probe vector sequence is reduced.

As a further consequence of excision or amplification of probes from vectors in construction of EST microarrays, the probes arrayed thereon often contain artificial sequence, derived from vector polylinker multiple cloning sites, at both 5' and 3' ends. The probes disposed upon the genome-derived single exon microarrays need have no such artificial sequence appended thereto.

As mentioned above, however, the exon-specific primers used to amplify putative exons can include artificial sequences, typically 5' to the exon-specific primer sequence, useful for "universal" (that is, independent of exon sequence) priming of subsequent amplification or sequencing reactions. When such "universal" 5' and/or 3' priming sequences are appended to the amplification primers, the probes disposed upon the genome-derived single exon microarray will include artificial sequence similar to that found in EST microarrays. However, the genome-derived single exon microarray of the present invention can be made without such sequences, and if so constructed, presents an even smaller amount of nonspecific sequence that would contribute to nonspecific hybridization.

Yet another consequence of typical use of cloned material as probes in EST microarrays is that such microarrays contain probes that result from cloning artifacts, such as chimeric molecules containing coding region of two separate genes. Derived from genomic material, typically not thereafter cloned, the probes of the genome-derived single exon microarrays of the present invention lack such cloning artifacts, and thus provide greater specificity of signal in gene expression measurements.

A further consequence of the cloned origin of probes on many EST microarrays is that the individual probes often have disparate sizes, which can cause the optimal hybridization stringency to vary among probes on a single microarray. In contrast, as discussed above, the probes arrayed on the genome-derived single exon microarrays of the present invention can readily be designed to have a narrow distribution in sizes, with the range of probe sizes no greater than about 10% of the average size, typically no greater than about 5% of the average probe size.

Because of their origin from fully- or partially-spliced message, probes disposed upon EST arrays will often include multiple exons. The percentage of such exon-spanning probes in an EST microarray can be calculated, on average, based upon the predicted number of exons/gene for the given species and the average length of the immobilized probes. For human genes, the near-complete sequence of human chromosome 22, Dunham et al., Nature 402(6761):489-95 (1999), predicts that human genes average 5.5 exons/gene. Even with probes of 200 - 500 bp, the vast majority of human EST microarray probes include more than one exon.

In contrast, by virtue of their origin from algorithmically identified exons in genomic sequence, the probes in the genome-derived single exon microarrays of the present invention can comprise individual exons, which provides the ability, as further discussed in commonly owned and copending U.S. patent application serial no. 09/632,366, filed August 3, 2000, incorporated herein by reference in its entirety, to detect and to characterize the expression of splice variants.

Although the presence of multiexon probes will not interfere with the ability to confirm expression of predicted exons in a first level screen, it is preferred that at least about 50%, typically at least about 60%, even more typically at least about 70% of probes disposed on the genome-derived microarray of the present invention consist of, or include, no more than one exon. In preferred embodiments, at least about 75%, more preferably at least about 80%, 85%, 90%, 95%, and even 99% of probes in the genome-derived microarrays of the present invention consist of, or include, no more than one exon.

Although, in the most preferred embodiments, at least about 95%, and even at least about 99% of probes in the genome-derived microarray consist of, or include, no more than one exon, we have found that our early bioinformatic parameters typically produce, at this stage of analysis, about 10% of probes that potentially contain two exons. We expect that some fraction of these probes will prove to encode only a single exon, and that further optimization of our bioinformatic approach will reduce the percentage of probes having more than one potential exon.

Further distinguishing the genome-derived single exon microarrays of the present invention from the EST arrays in the art, the exons that are represented in EST microarrays are often biased toward the 3' or 5' end of their respective genes, since sequencing strategies used for EST identification are so biased. In contrast, no such 3' or 5' bias necessarily inheres in the selection of exons for disposition on the genome-derived single exon microarrays of the present invention.

Conversely, the probes provided on the genome-derived single exon microarrays of the present invention typically, but need not necessarily, include intronic and/or intergenic sequence that is absent from EST microarrays, which are derived from mature mRNA. As above mentioned, such inclusion, although not mandatory, is advantageous, particularly in use of the probes for detection of alternative splice events. Typically, therefore, at least about 50%, more typically at least about 60%, and even more typically at least about 70% of the exon-including probes on the genome-derived single exon microarrays of the present invention include sequence drawn from noncoding regions. In some embodiments, at least about 80%, more typically at least about 85%, 90%, 91%, 92%, 93%, 94%, 95%, 96%, 97%, 98%, and even 99% or more of exon-including probes on the genome-derived single exon microarrays of the present invention will include sequence drawn from noncoding regions.

The genome-derived single exon microarrays of the present invention are also quite different from in situ synthesis microarrays, where probe size is severely constrained by limitations of the photolithographic or other in situ synthesis processes.

Typically, probes arrayed on in situ synthesis microarrays are limited to a maximum of about 25 bp. As a well known consequence, hybridization to such chips must be performed at low stringency. In order, therefore, to achieve unambiguous sequence-specific hybridization results, the in situ synthesis microarray requires substantial redundancy, with concomitant programmed arraying for each probe of probe analogues with altered (i.e., mismatched) sequence.

In contrast, the longer probe length of the genome-derived single exon microarrays of the present invention allows much higher stringency hybridization and wash. Typically, therefore, exon-including probes on the genome-derived single exon microarrays of the present invention average at least about 100 bp, more typically at least about 200 bp, preferably at least about 250 bp, even more preferably about 300 bp, 400 bp, or in preferred embodiments, at least about 500 bp in length. By obviating the need for substantial probe redundancy, this approach permits a higher density of probes for discrete exons or genes to be arrayed on the microarrays of the present invention than can be achieved for in situ synthesis microarrays.

A further distinction is that the probes in in situ synthesis microarrays typically are covalently linked to the substrate surface. In contrast, the probes disposed on the genome-derived microarray of the present invention typically are, but need not necessarily be, bound noncovalently to the substrate.

Furthermore, the short probe size on in situ microarrays causes large percentage differences in the melting temperature of probes hybridized to their complementary target sequence, and thus causes large percentage differences in the theoretically optimum stringency across the array as a whole.

In contrast, the larger probe size in the microarrays of the present invention create lower percentage differences in melting temperature across the range of arrayed probes.

A further significant advantage of the microarrays of the present invention over in situ synthesized arrays is that the quality of each individual probe can be confirmed before deposition. In contrast, the quality of probes cannot be assessed on a probe-by-probe basis for the in situ synthesized microarrays presently being used.

The genome-derived single exon microarrays of the present invention are also distinguished over, and present substantial benefits over, the genome-derived microarrays from lower eukaryotes such as yeast. See, e.g., Lashkari et al., Proc. Natl. Acad. Sci. USA 94:13057-13062 (1997).

Only about 220 - 250 of the 6100 or so nuclear genes in Saccharomyces cerevisiae — that is, only about 4 to 5% —have standard, spliceosomal, introns, Lopez et al., Nucl. Acids Res. 28:85-86 (2000); Spingola et al., RNA 5(2):221-34 (1999), permitting the ready amplification and disposition of single-exon amplicons on such microarray without the requirement for antecedent use of gene prediction and/or comparative sequence analyses.

A significant aspect of the present invention is the ability to identify and to confirm expression of predicted coding regions in genomic sequence drawn from eukaryotic organisms that have a higher percentage of genes having introns than do yeast such as Saccharomyces cerevisiae, particularly in genomic sequence drawn from eukaryotes in which at least about 10%, typically at least about 20%, more typically at least about 50% of protein-encoding genes have introns. In preferred embodiments, the methods and apparatus of the present invention are used to identify and confirm expression of exons of novel genes from genomic sequence of eukaryotes in which the average number of introns per gene is at least about one, more typically at least about two, even more typically at least about three or more.

After the physical substrate is prepared, experimental verification of predicted function is performed.

In a preferred embodiment of the present invention, where the function sought to be identified in genomic sequence is protein coding, experimental verification is performed by measuring expression of the putative exons, typically through nucleic acid hybridization experiments, and in particularly preferred embodiments, through hybridization to genome-derived single exon microarrays prepared as above described.

Expression is conveniently measured and reported for each probe in the microarray both as a signal intensity and as a ratio of the expression measured relative to a control, according to techniques well known in the microarray art, reviewed in Schena (ed.), DNA Microarrays : A Practical Approach (Practical Approach Series), Oxford University Press (1999) (ISBN: 0199637768); Nature Genet. 21(1)(suppl):1-60 (1999); Schena (ed.), Microarray Biochip: Tools and Technology, Eaton Publishing Company/BioTechniques Books Division (2000) (ISBN: 1881299376), the disclosures of which are incorporated herein by reference in their entireties. See also Example 2, infra. The mRNA source for the reference (control) used to calculate expression ratios can be heterogeneous, as from a pool of multiple tissues and/or cell types or, alternatively, can be drawn from a homogeneous mRNA source, such as a single cultured cell-type.

In Examples 1 and 2, infra, we used a pool of 10 tissues/cell types as control. We have since observed that almost every probe that demonstrates expression in the control pool can readily be shown to be expressed in HeLa cells. Since use of a pooled control might mask subtle alternative splice events, we have used HeLa as the source of control message in more recent experiments.

mRNA can be prepared by standard techniques, Short Protocols in Molecular Biology : A Compendium of Methods from Current Protocols in Molecular Biology, Ausubel et al. (eds.), 4^{th} edition (April 1999), John Wiley & Sons (ISBN: 047132938X) and Maniatis et al., Molecular Cloning : A Laboratory Manual, 2^{nd} edition (December 1989), Cold Spring Harbor Laboratory Press (ISBN: 0879693096), the disclosures of which are incorporated herein by reference in their entireties, or purchased commercially. The mRNA is then typically reverse-transcribed in the presence of labeled nucleotides: the index source (that in which expression is desired to be measured) is reverse transcribed in the presence of nucleotides labeled with a first label, typically a fluorophore (equivalently denominated fluorochrome; fluor; fluorescent dye); the reference source is reverse transcribed in the presence of a second label, typically a fluorophore, typically fluorometrically-distinguishable from the first label. As further described in Example 2, infra, Cy3 and Cy5 dyes prove particularly useful in these methods. After partial purification of the index and reference targets, hybridization to the probe array is conducted according to standard techniques, typically under a coverslip or in an automatic slide processing unit.

After wash, microarrays are conveniently scanned using a commercial microarray scanning device, such as a Gen3 or Avalanche Scanner (Molecular Dynamics, Sunnyvale, CA). Data on expression is then passed, with or without interim storage, to process 500, where the results for each probe are related to the original sequence.

Often, hybridization of target material to the genome-derived single exon microarray will identify certain of the probes thereon as of particular interest. Thus, it is often desirable that the user be able readily to obtain sufficient quantities of an individual probe, either for subsequent arrayed deposition upon an additional support substrate, often as part of a microarray having a plurality of probes so identified, or alternatively or additionally as a solitary solid-phase or solution-phase probe for further use.

Thus, in another aspect, the present invention provides compositions and kits for the ready production of nucleic acids identical in sequence to, or substantially identical in sequence to, probes on the genome-derived single exon microarrays of the present invention.

In one embodiment, the invention provides individual single exon probes in the form of substantially isolated and purified nucleic acid. In one such embodiment the probe is provided in quantity sufficient to perform a hybridization reaction.

When provided in quantity sufficient to perform a hybridization reaction, the probe can be in any form directly hybridizable to the target that contains the probe's exon (or its complement), such as double stranded DNA, single-stranded DNA complementary to the target, single-stranded RNA complementary to the target, or chimeric DNA/RNA molecules so hybridizable.

The nucleic acid can alternatively or additionally include either nonnative nucleotides, alternative internucleotide linkages, or both, so long as complementary binding can be obtained. For example, probes can include phosphorothioates, methylphosphonates, morpholino analogs, and peptide nucleic acids (PNA), as are described, inter alia, in U.S. Patent Nos. 5,142,047; 5,235,033; 5,166,315; 5,217,866; 5,184,444; 5,861,250; international patent applications nos. WO 93/25706, and in Science 254:1497 (1991); J. Am. Chem. Soc. 114:9677 (1992); J. Am. Chem. Soc. 144:1895 (1992); J. Chem. Soc. Chem. Comm. 800 (1993); Proc. Nat. Acad. Sci. USA 90:1667 (1993); Intercept Ltd. 325 (1992); J. Am. Chem. Soc. 114:9677 (1992); Nucleic Acids Res. 21:197 (1993); J. Chem. Soc. Chem. Commun. 518 (1993); Anti-Cancer Drug Design 8:53 (1993); Nucleic Acids Res. 21:2103 (1993); Org. Proc. Prep. 25:457 (1993); CRC Press 363 (1992); J. Chem. Soc. Chem. Commun. 9:800 (1993); J. Am. Chem. Soc. 115:6477 (1993); Nature 365:566 (1993); WO 92/20702; and WO 92/20703, the disclosures of which are incorporated herein by reference.

Usefully, however, such probes are instead provided in a form and quantity suitable for amplification, such as by PCR. Although PCR is conveniently used, other amplification approaches can be used as well, such as rolling circle amplification, as is described, inter alia, in U.S. Patent Nos. 5,854,033 and 5,714,320 and international patent publications WO97/19193 and WO 00/15779, the disclosures of which are incorporated herein by reference in their entireties. As is well understood, where the probes are to be provided in a form suitable for amplification, the range of nucleic acid analogues and/or internucleotide linkages will be constrained by the requirements and nature of the amplification enzyme.

Where the probe is to be provided in form suitable for amplification, the quantity need not be sufficient for direct hybridization for gene expression analysis, and need be sufficient only to function as an amplification template, typically at least about 1 pg, more typically at least about 10 pg, and usually at least about 100 pg or more.

Each discrete amplifiable probe can also be packaged with amplification primers, either in a single composition that comprises probe template and primers, or in a kit that comprises such primers separately packaged therefrom. As above mentioned, the exon-specific 5' primers used for genomic amplification can have a first common sequence added thereto, and the exon-specific 3' primers used for genomic amplification can have a second, different, common sequence added thereto, thus permitting, in this embodiment, the use of a single set of 5' and 3' primers to amplify any one of the probes. The probe composition and/or kit can also include buffers, enzyme, etc., required to effect amplification.

In another embodiment, only amplification primers are provided. The primers are sufficient to permit generation of the single exon probe by amplification from genomic DNA, which can be provided by the user.

As mentioned above, when intended for use on a genome-derived single exon microarray of the present invention, the genome-derived single exon probes of the present invention will typically average at least about 75 - 100 bp, more typically at least about 200 bp, preferably at least about 250 bp, even more preferably about 300 bp, 400 bp or in preferred embodiments, at least about 500 bp in length, including (and typically, but not necessarily centered about) the exon. Furthermore, when intended for use on a genome-derived single exon microarray of the present invention, the genome-derived single exon probes of the present invention will typically not contain a detectable label.

When intended for use in solution phase hybridization, however —that is, for use in a hybridization reaction in which the probe is not first bound to a support substrate (although the target may indeed be so bound) — length constraints that are imposed in microarray-based hybridization approaches will be relaxed, and such probes will typically be labeled.

In such case, the only functional constraint that dictates the minimum size of such probe is that each such probe must be capable of specifically identifying in a hybridization reaction the exon from which it is drawn. In theory, a probe of as little as 17 nucleotides is capable of uniquely identifying its cognate sequence in the human genome. For hybridization to expressed message — a subset of target sequence that is much reduced in complexity as compared to genomic sequence — even fewer nucleotides are required for specificity.

Therefore, the probes of the present invention can include as few as 20 bp of exon, typically at least about 25 bp of exon, more typically at least about 50 bp or exon, or more. The minimum amount of exon required to be included in the probe of the present invention in order to provide specific signal in either solution phase or microarray-based hybridizations can readily be determined by routine experimentation using standard high stringency conditions.

Such high stringency conditions are described, inter alia, in Short Protocols in Molecular Biology : A Compendium of Methods from Current Protocols in Molecular Biology, Ausubel et al. (eds.), 4th edition (April 1999), John Wiley & Sons (ISBN: 047132938X) and Maniatis et al., Molecular Cloning : A Laboratory Manual, 2nd edition (December 1989), Cold Spring Harbor Laboratory Press (ISBN: 0879693096), the disclosures of which are incorporated herein by reference in their entireties.

For microarray-based hybridization, standard high stringency conditions can usefully be 50% formamide, 5X SSC, 0.2 μg/μl poly(dA), 0.2 μg/μl human cot1 DNA, and 0.5 % SDS, in a humid oven at 42ºC overnight, followed by successive washes of the microarray in 1X SSC, 0.2% SDS at 55ºC for 5 minutes, and then 0.1X SSC, 0.2% SDS, at 55ºC for 20 minutes.

For solution phase hybridization, standard high stringency conditions can usefully be aqueous hybridization at 65ºC in 6X SSC.

Lower stringency conditions, suitable for cross-hybridization to mRNA encoding structurally- and functionally-related proteins, can usefully be the same as the high stringency conditions but with reduction in temperature for hybridization and washing to room temperature (approximately 25ºC).

When intended for use in solution phase hybridization, the maximum size of the single exon probes of the present invention is dictated by the proximity of other exons in genomic DNA: although each single exon probe can include intergenic and/or intronic material contiguous to the exon in the human genome, each probe of the present invention will typically include portions of only one exon.

Thus, each single exon probe will include no more than about 25 kb of contiguous genomic sequence, more typically no more than about 20 kb of contiguous genomic sequence, more usually no more than about 15 kb, even more usually no more than about 10 kb. Usually, probes that are maximally about 5 kb will be used, more typically no more than about 3 kb.

It will be appreciated that single stranded probes must be complementary in sequence to the target; it is well within the skill in the art to determine such complementary sequence and the need therefor. It will further be understood that double stranded probes can be used in both solution-phase hybridization and microarray-based hybridization if suitably denatured. Thus, it is an aspect of the present invention to provide single-stranded nucleic acid probes that have sequence complementary to those described herein above and below, and double-stranded probes one strand of which has sequence complementary to the probes described herein.

As mentioned above, the probes can, but need not, contain intergenic and/or intronic material that flanks the exon, on one or both sides, in the same linear relationship to the exon that the intergenic and/or intronic material bears to the exon in genomic DNA. The probes typically do not, however, contain nucleic acid derived from more than one expressed exon.

And when intended for use in solution hybridization, the probes of the present invention can usefully have detectable labels. Nucleic acid labels are well known in the art, and include, inter alia, radioactive labels, such as ³H, ³²P, ³³P, ³⁵S, ¹²⁵I, ¹³¹I; fluorescent labels, such as Cy3, Cy5, Cy5.5, Cy7, SYBR®Green and other labels described in Haugland, Handbook of Fluorescent Probes and Research Chemicals, 7th ed., Molecular Probes Inc., Eugene, OR (2000), or fluorescence resonance energy transfer tandem conjugates thereof; labels suitable for chemiluminescent and/or enhanced chemiluminescent detection; labels suitable for ESR and NMR detection; quantum dots; and labels that include one member of a specific binding pair, such as biotin, digoxigenin, or the like.

The probes, either in quantity sufficient for hybridization or sufficient for amplification, can be provided in individual vials or containers, and can be provided dry (e.g., lyophilized), or solvated. If solvated, the solution can usefully include buffers and salts as desired for hybridization and/or amplification. Furthermore, if desired to be spotted on a microarray, the probes can usefully be provided in a solution of chaotropic agent to facilitate adherence to the microarray support substrate.

Alternatively, such probes can usefully be packaged as a plurality of such individual genome-derived single exon probes.

In one embodiment of this aspect, a small quantity of each probe is disposed, typically without attachment to substrate, in a spatially-addressable ordered set, typically one per well of a microtiter dish. Although a 96 well microtiter plate can be used, greater efficiency is obtained using higher density arrays, such as are provided by microtiter plates having 384, 864, 1536, 3456, 6144, or 9600 wells. And although microtiter plates having physical depressions (wells) are conveniently used, any device that permits addressable withdrawal of reagent from fluidly-noncommunicating areas can be used.

Each of the probes of the ordered set can be provided in any of the forms that are described above with respect to the probes as individually packaged.

As above mentioned, the exon-specific 5' primer used for genomic amplification can have a first common sequence added thereto, and the exon-specific 3' primers used for genomic amplification can have a second, different, common sequence added thereto, thus permitting, in certain embodiments, the use of a single set of 5' and 3' primers to amplify any one of the probes from the amplifiable ordered set.

Such collections of genome-derived single exon probes can usefully include a plurality of probes chosen for a common attribute, such as common expression in a given tissue, cell type, developmental stage, disease state, or the like.

In such defined subsets, typically at least 50% of the probes will have the common attribute, such as expression in the defined tissue or cell type. More typically, at least about 60% of the probes will be expressed in the defined tissue, even more typically at least about 75%, and preferably at least about 80%, 85%, or, in preferred embodiments, at least about 90%, and even 95% or more of the probes will have the common attribute, such as expression in the defined tissue or cell type.

Analogously, the invention provides, in another aspect, genome-derived single-exon nucleic acid microarrays having a plurality of probes chosen for a common attribute, such as common expression in a given tissue, cell type, developmental stage, disease state, or the like.

These "subset-defined" genome-derived single exon microarrays can be distinguished from the "first iteration" genome-derived single exon microarrays of the present invention, i.e., from those that are used to confirm expression of predicted exons, by the percentage of probes that are known to have a common attribute, such as expression in a defined tissue or cell type. On such "subset-defined" microarrays, typically at least 50% of the probes will have the common attribute, typically expression in the defined tissue or cell type. More typically, at least about 60% of the probes will be expressed in the defined tissue, even more typically at least about 75%, and preferably at least about 80%, 85%, or, in preferred embodiments, at least about 90%, and even 95% or more of the probes will have the common attribute, such as expression in the defined tissue or cell type.

When used for gene expression analysis, the "defined subset" genome-derived single exon microarrays provide greater physical informational density than do the genome-derived single exon microarrays that have lower percentages of probes known to be expressed commonly in the tested tissue. At a fixed probe density, for example, a given microarray surface area of the defined subset genome-derived single exon microarray can yield a greater number of expression measurements. Alternatively, at a given probe density, the same number of expression measurements can be obtained from a smaller substrate surface area. Alternatively, at a fixed probe density and fixed surface area, probes can be provided redundantly, providing greater reliability in signal measurement for any given probe. Furthermore, with a higher percentage of probes known to be expressed in the assayed tissue, the dynamic range of the detection means can be adjusted to reveal finer levels of discrimination among the levels of expression.

In another aspect of the present invention, a genome-derived single-exon microarray is packaged together with an addressable set of individual probes, the set of individual probes including at least a subset of the probes on the microarray. In alternative embodiments, the ordered set of amplifiable probes is packaged separately from the genome-derived single exon microarray.

In some embodiments, the microarray and/or ordered probe set are further packaged with recorded media that provide probe identification and addressing information, and that can additionally contain annotation information, such as gene expression data. Such recorded media can be packaged with the microarray, with the ordered probe set, or with both.

If the microarray is constructed on a substrate that incorporates recordable media, such as is described in international patent application no. WO 98/12559, entitled "Spatially addressable combinatorial chemical arrays in CD-ROM format," incorporated herein by reference in its entirety, then separate packaging of the genome-derived single exon microarray and the bioinformatic information is not required.

Although the use of high density genome-derived microarrays on solid planar substrates is presently a preferred approach for the physical confirmation and characterization of the expression of sequences predicted to encode protein, other types of microarrays, as well as lower density macro arrays, can also be used.

Experimental verification in process 400 of the function predicted from genomic sequence in process 200 can be bioinformatic, rather than, or additional to, physical verification.

Where the function desired to be identified is protein coding, the predicted exons can be compared bioinformatically to sequences known or suspected of being expressed.

Thus, the sequences output from process 300 (or process 200), can be used to query expression databases, such as EST databases, SNP ("single nucleotide polymorphism") databases, known cDNA and mRNA sequences, SAGE ("serial analysis of gene expression") databases, and more generalized sequence databases that allow query for expressed sequences. Such query can be done by any sequence query algorithm, such as BLAST ("basic local alignment search tool"). The results of such query — including information on identical sequences and information on nonidentical sequences that have diffuse or focal regions of sequence homology to the query sequence — can then be passed directly to process 500, or used to inform analyses subsequently undertaken in process 200, process 300, or process 400.

Experimental data, whether obtained by physical or bioinformatic assay in process 400, is passed to process 500 where it is usefully related to the sequence data itself, a process colloquially termed "annotation". Such annotation can be done using any technique that usefully relates the functional information to the sequence, as, for example, by incorporating the functional data into the record itself, by linking records in a hierarchical or relational database, by linking to external databases, or by a combination thereof. Such database techniques are well within the skill in the art.

The annotated sequence data can be stored locally, uploaded to genomic sequence database 100, and/or displayed 800.

The methods and apparatus of the present invention rapidly produce functional information from genomic sequence. We have, for example, used the methods and apparatus of the present invention to identify over 15,000 exons in human genomic sequence whose expression we have confirmed in at least one human tissue or cell type. Fully two-thirds of the exons belong to genes that were not then represented in existing public expression (EST, cDNA) databases. We have also used these single exon probes to identify alternative splice events in novel genes.

Coupled with the escalating pace at which sequence now accumulates, the ability rapidly to identify and confirm the function of regions of genomic DNA provided by the present invention produces a need for methods of displaying the information in meaningful ways. It is, therefore, another aspect of the present invention to provide means for displaying annotated sequence, and in particular for displaying sequence annotated according to the methods and apparatus of the present invention. Further, such display can be used as a preferred graphical user interface for electronic search, query, and analysis of such annotated sequence.

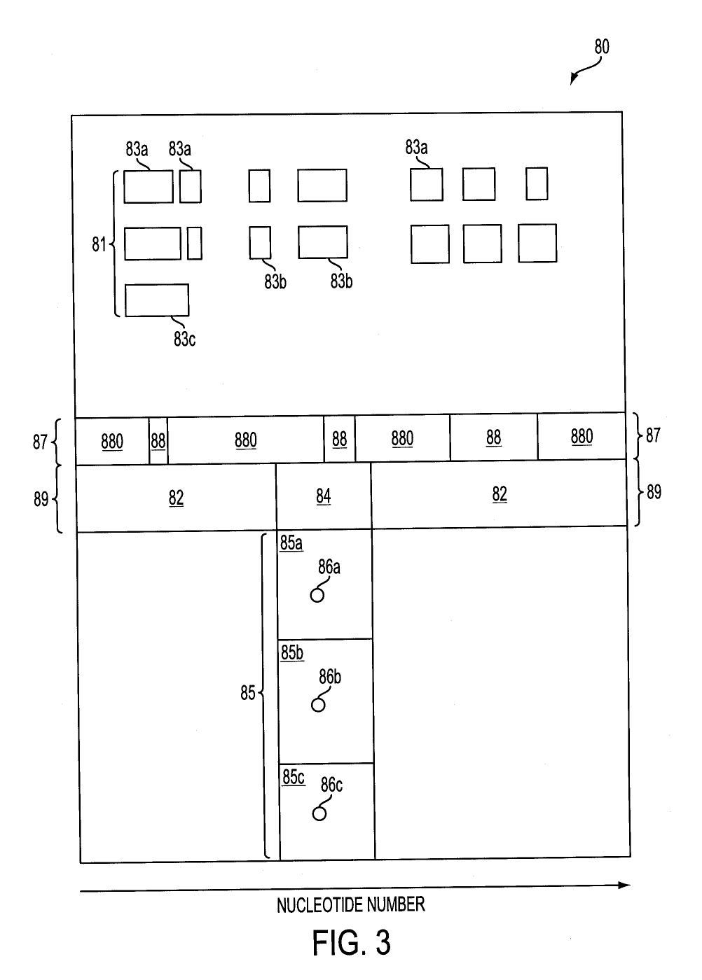

FIG. 3 schematizes visual display 80 presenting a single genomic sequence annotated according to the present invention. Because of its nominal resemblance to artistic works of Piet Mondrian, visual display 80 is alternatively described herein as a "Mondrian".

Each of the visual elements of display 80 is aligned with respect to the genomic sequence being annotated (the "annotated sequence"). Given the number of nucleotides typically represented in an annotated sequence, representation of individual nucleotides would rarely be readable in hard copy output of display 80. Typically, therefore, the annotated sequence is schematized as rectangle 89, extending from the left border of display 80 to its right border. By convention herein, the left border of rectangle 89 represents the first nucleotide of the sequence and the right border of rectangle 89 represents the last nucleotide of the sequence.

As further discussed below, however, the Mondrian visual display of annotated sequence can serve as a convenient graphical user interface for computerized representation, analysis, and query of information stored electronically. For such use, the individual nucleotides can conveniently be linked to the X axis coordinate of rectangle 89. This permits the annotated sequence at any point within rectangle 89 readily to be viewed, either automatically — for example, by time-delayed appearance of a small overlaid window ("tool tip") upon movement of a cursor or other pointer over rectangle 89 — or through user intervention, as by clicking a mouse or other pointing device at a point in rectangle 89.

Visual display 80 is generated after user specification of the genomic sequence to be displayed. Such specification can consist of or include an accession number for a single clone (e.g., a single BAC accessioned into GenBank), wherein the starting and stopping nucleotides are thus absolutely identified, or alternatively can consist of or include an anchor or fulcrum point about which a chosen range of sequence is anchored, thus providing relative endpoints for the sequence to be displayed. For example, the user can anchor such a range about a given chromosomal map location, gene name, or even a sequence returned by query for similarity or identity to an input query sequence. When visual display 80 is used as a graphical user interface to computerized data, additional control over the first and last displayed nucleotide will typically be dynamically selectable, as by use of standard zooming and/or selection tools.

Field 81 of visual display 80 is used to present the output from process 200, that is, to present the bioinformatic prediction of those sequences having the desired function within the genomic sequence. Functional sequences are typically indicated by at least one rectangle 83 (83a, 83b, 83c), the left and right borders of which respectively indicate, by their X-axis coordinates, the starting and ending nucleotides of the region predicted to have function.

Where a single bioinformatic method or approach identifies a plurality of regions having the desired function, a plurality of rectangles 83 is disposed horizontally in field 81. Where multiple methods and/or approaches are used to identify function, each such method and/or approach can be represented by its own series of horizontally disposed rectangles 83, each such horizontally disposed series of rectangles offset vertically from those representing the results of the other methods and approaches.

Thus, rectangles 83a in FIG. 3 represent the functional predictions of a first method of a first approach for predicting function, rectangles 83b represent the functional predictions of a second method and/or second approach for predicting that function, and rectangles 83c represent the predictions of a third method and/or approach.

Where the function desired to be identified is protein coding, field 81 is used to present the bioinformatic prediction of sequences encoding protein. For example, rectangles 83a can represent the results from GRAIL or GRAIL II, rectangles 83b can represent the results from GENEFINDER, and rectangles 83c can represent the results from DICTION.

Optionally, and preferably, rectangles 83 collectively representing predictions of a single method and/or approach are identically colored and/or textured, and are distinguishable from the color and/or texture used for a different method and/or approach.

Alternatively, or in addition, the color, hue, density, or texture of rectangles 83 can be used further to report a measure of the bioinformatic reliability of the prediction. For example, many gene prediction programs will report a measure of the reliability of prediction. Thus, increasing degrees of such reliability can be indicated, e.g., by increasing density of shading. Where display 80 is used as a graphical user interface, such measures of reliability, and indeed all other results output by the program, can additionally or alternatively be made accessible through linkage from individual rectangles 83, as by time-delayed window ("tool tip" window), or by pointer (e.g., mouse)-activated link.

As above described, increased predictive reliability can be achieved by requiring consensus among methods and/or approaches to determining function. Thus, field 81 can include a horizontal series of rectangles 83 that indicate one or more degrees of consensus in predictions of function, including the combined length of the separately predicted exons that overlap in frame.

Although FIG. 3 shows three series of horizontally disposed rectangles in field 81, display 80 can include as few as one such series of rectangles and as many as can discriminably be displayed, depending upon the number of methods and/or approaches used to predict a given function. For example, addition of a fourth gene prediction program, such as GENSCAN (http://genes.mit.edu/GENSCANinfo.html), to the three gene prediction programs used in our first experiments (GRAIL, GENEFINDER, DICTION) would be accommodated by a fourth series of rectangles disposed horizontally in field 81, but offset vertically from rectangles 81a, 81b, and 81c.

Furthermore, field 81 can be used to show predictions of a plurality of different functions. However, the increased visual complexity occasioned by such display makes more useful the ability of the user to select a single function for display. When display 80 is used as a graphical user interface for computer query and analysis, such function can usefully be indicated and user-selectable, as by a series of graphical buttons or tabs (not shown in FIG. 3).

Rectangle 89 is shown in FIG. 3 as including interposed rectangle 84. Rectangle 84 represents the portion of annotated sequence for which predicted functional information has been assayed physically, with the starting and ending nucleotides of the assayed material indicated by the X axis coordinates of the left and right borders of rectangle 84. Rectangle 85, with optional inclusive circles 86 (86a, 86b, and 86c) displays the results of such physical assay.

Although a single rectangle 84 is shown in FIG. 3, physical assay is not limited to just one region of annotated genomic sequence. It is expected that an increasing percentage of regions predicted to have function by process 200 will be assayed physically, and that display 80 will accordingly, for any given genomic sequence, have an increasing number of rectangles 84 and 85, representing an increased density of sequence annotation. For example, for purposes of generating exon-specific probes for alternative splice detection, it is preferred that a plurality of exons, preferably all of the exons, that commonly belong to a single gene will be assayed experimentally for expression; accordingly, display 80 will have, for the genomic sequence encompassing such exons, a series of rectangles 84 and 85 for each of the assayed exons.

Where the function desired to be identified is protein coding, rectangle 84 identifies the sequence of the probe used to measure expression. In embodiments of the present invention where expression is measured using genome-derived single exon microarrays, rectangle 84 identifies the sequence included within the probe immobilized on the solid support surface of the microarray. As noted supra, such probe will often include a small amount of additional, synthetic, material incorporated during amplification and designed to permit reamplification of the probe, which sequence is typically not shown in display 80.

Rectangle 87 is used to present the results of bioinformatic assay of the genomic sequence. For example, where the function desired to be identified is protein coding, process 400 can include bioinformatic query of expression databases with the sequences predicted in process 200 to encode exons. And as above discussed, because bioinformatic assay presents fewer constraints than does physical assay, often the entire output of process 200 can be used for such assay, without further subsetting thereof by process 300. Therefore, rectangle 87 typically need not have separate indicators therein of regions submitted for bioinformatic assay; that is, rectangle 87 typically need not have regions therein analogous to rectangles 84 within rectangle 89.

Rectangle 87 as shown in FIG. 3 includes smaller rectangles 880 and 88. Rectangles 880 indicate regions that returned a positive result in the bioinformatic assay, with rectangles 88 representing regions that did not return such positive results. Where the function desired to be predicted and displayed is protein coding, rectangles 880 indicate regions of the predicted exons that identify sequence with significant similarity in expression databases, such as EST, SNP, SAGE databases, with rectangles 88 indicating genes novel over those identified in existing expression data bases.

Rectangles 880 can further indicate, through color, shading, texture, or the like, additional information obtained from bioinformatic assay.

For example, where the function assayed and displayed is protein coding, the degree of shading of rectangles 880 can be used to represent the degree of sequence similarity found upon query of expression databases. The number of levels of discrimination can be as few as two (identity, and similarity, where similarity has a user-selectable lower threshold). Alternatively, as many different levels of discrimination can be indicated as can visually be discriminated.

Where display 80 is used as a graphical user interface, rectangles 880 can additionally provide links directly to the sequences identified by the query of expression databases, and/or statistical summaries thereof. As with each of the precedingly-discussed uses of display 80 as a graphical user interface, it should be understood that the information accessed via display 80 need not be resident on the computer presenting such display, which often will be serving as a client, with the linked information resident on one or more remotely located servers.

Rectangle 85 displays the results of physical assay of the sequence delimited by its left and right borders.

Rectangle 85 can consist of a single rectangle, thus indicating a single assay, or alternatively, and increasingly typically, will consist of a series of rectangles (85a, 85b, 85c) indicating separate physical assays of the same sequence.

Where the function assayed is gene expression, and where gene expression is assayed as herein described using simultaneous two-color fluorescent detection of hybridization to genome-derived single exon microarrays, individual rectangles 85 can be colored to indicate the degree of expression relative to control. Conveniently, shades of green can be used to depict expression in the sample over control values, and shades of red used to depict expression less than control, corresponding to the spectra of the Cy3 and Cy5 dyes conventionally used for respective labeling thereof. Additional functional information can be provided in the form of circles 86 (86a, 86b, 86c), where the diameter of the circle can be used to indicate a parameter different from that set forth in rectangle 85. For example, where the annotated functions are the distribution of expression of the one or more predicted exons, rectangle 85 can report expression relative to control and circle 86 can be used to report signal intensity. As discussed infra, such relative expression (expression ratio) and absolute expression (signal intensity) can be expressed using normalized values.

Where display 80 is used as a graphical user interface, rectangle 85 can be used as a link to further information about the assay. For example, where the assay is one for gene expression, each rectangle 85 can be used to link to information about the source of the hybridized mRNA, the identity of the control, raw or processed data from the microarray scan, or the like.

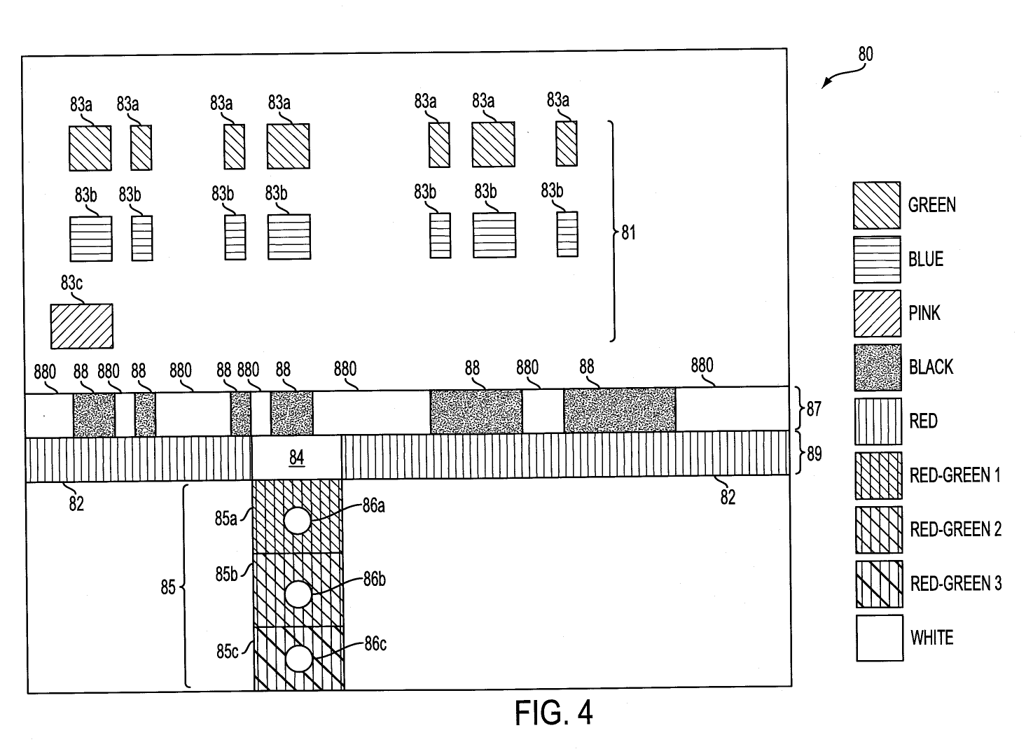

For purposes of illustration only, FIG. 4 shows an embodiment of display 80 showing typical color conventions when hypothetical genomic sequence is annotated with exon-specific expression data. As would of course readily be understood, the color choice is arbitrary, and alternative colors can be used.

In this typical presentation, BAC sequence ("Chip seq.") 89 is presented in red, with the physically assayed region thereof (corresponding to rectangle 84 in FIG. 3) shown in white. Algorithmic gene predictions are shown in field 81, with predictions by GRAIL shown in green, predictions by GENEFINDER shown in blue, and predictions by DICTION shown in pink. Within rectangle 87, regions of sequence that, when used to query expression databases, return identical or similar sequences ("EST hit") are shown as white rectangles (corresponding to rectangles 880 in FIG. 3), gray indicates low homology, and black indicates unknowns (where black and gray would correspond to rectangles 88 in FIG. 3).

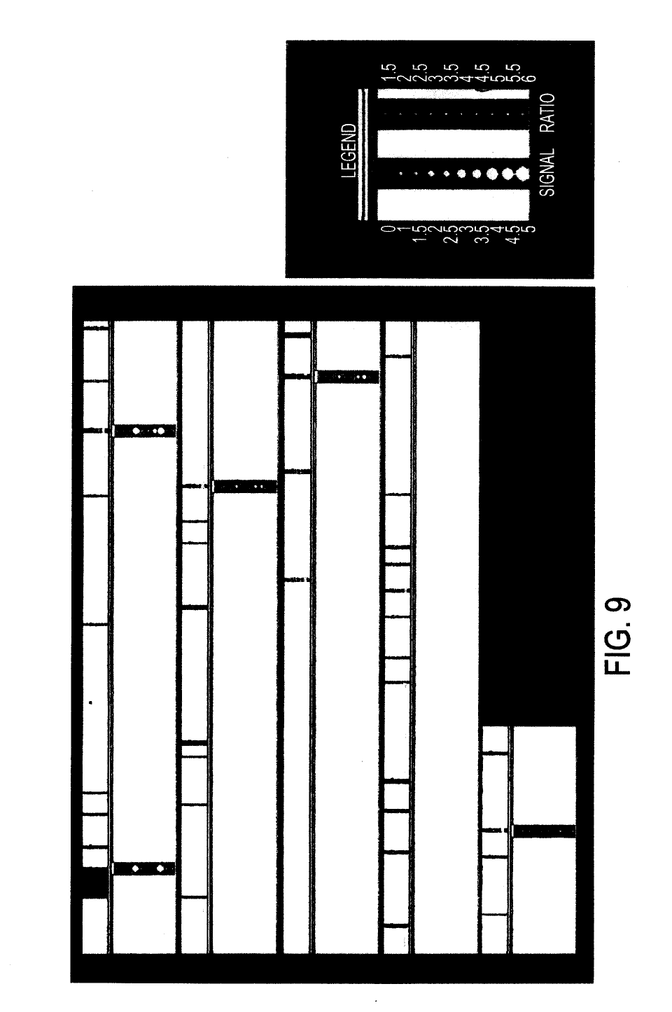

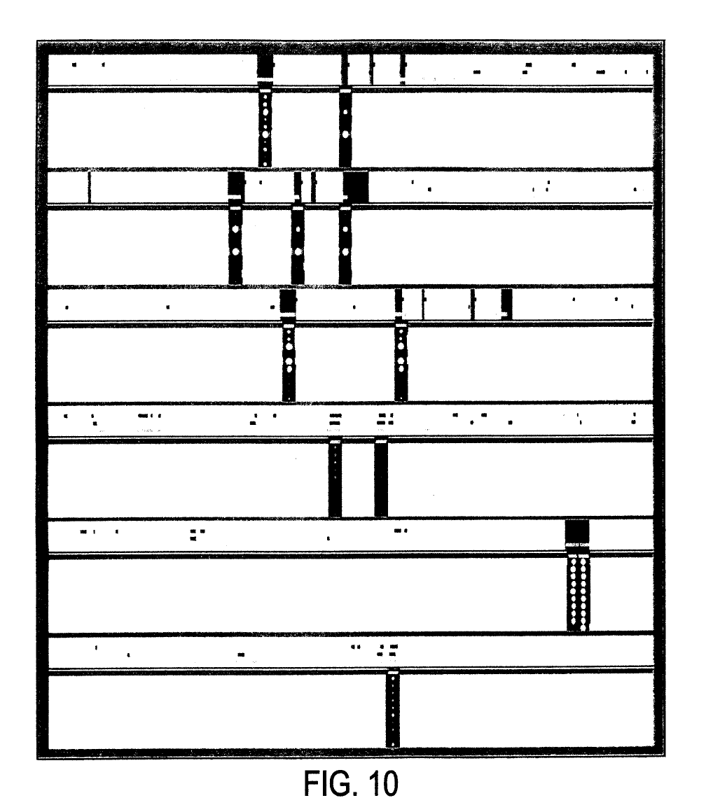

Although FIGS. 3 and 4 show a single stretch of sequence, uninterrupted from left to right, longer sequences are usefully represented by vertical stacking of such individual Mondrians, as shown in FIGS. 9 and 10.

Using our visual display tool, the Mondrian, we have found that consensus in the pattern of expression of individual exons is a powerful means for identifying exons that commonly belong to a single gene. It is, therefore, another aspect of the present invention to provide methods, including methods based upon visual display, for associating exons that commonly belong to a single gene using, as the criterion for association, consensus in their patterns of expression in a plurality of tissues and/or cell types.

As further discussed in Example 3, FIG. 9 presents a Mondrian of BAC AC008172 (bases 25,000 to 130,000 shown), containing the carbamyl phosphate synthetase gene (AF154830.1), the sequence and structure of which has previously been reported. Purple background within the region shown as field 81 in FIG. 3 indicates all 37 known exons for this gene.

As can be seen, GRAIL II successfully identified 27 of the known exons (73%), GENEFINDER successfully identified 37 of the known exons (100%), while DICTION identified 7 of the known exons (19%).

Seven of the predicted exons were selected for physical assay, of which 5 successfully amplified by PCR and were sequenced. These five exons were all found to be from the same gene, the carbamyl phosphate synthetase gene (AF154830.1).

The five exons were arrayed and gene expression measured across 10 tissues. As is readily seen by visual inspection of the resulting Mondrian (FIG. 9), the five single-exon probes report identical expression ratio patterns: each exon is expressed above control (i.e., in green) in the tissues represented by the fourth, seventh, and eighth rectangles (corresponding to rectangles 85 in FIG. 3) and is expressed at or below control in the remaining tissues.

Of course, an exon that is removed or truncated by alternatively splicing in one of the assayed tissues would produce a variant expression pattern. For purposes of associating exons as belonging commonly to a single gene, however, a consensus among assayed tissues would still identify the exon as presumptively belonging to the same gene.

The methods of this aspect of the invention can, and typically will, be automated. For example, WO 99/58720, incorporated herein by reference in its entirety, describes algorithms for ordering the relatedness of a plurality of multidimensional expression data sets. The methods set forth therein can readily be adapted to ordering the relatedness of data sets, wherein each data set comprises expression ratios of an individual exon across a plurality of tissues and cell types, permitting exons with related, but not necessarily identical, patterns of expression to be classified as belonging to a common gene.

The following examples are offered by way of illustration and not by way of limitation.

EXAMPLE 1

Preparation of Single Exon Microarrays from Exons Predicted in Human Genomic Sequence

Bioinformatics Results

All human BAC sequences in fewer than 10 pieces that had been accessioned in a five month period immediately preceding this study were downloaded from GenBank. This corresponds to ≈2200 clones, totaling ≈350 MB of sequence, or approximately 10% of the human genome.

After masking repetitive elements using the program CROSS-MATCH, the sequence was analyzed for open reading frames using three separate gene finding programs. The three programs predict genes using independent algorithmic methods developed on independent training sets: GRAIL uses a neural network, GENEFINDER uses a hidden Markoff model, and DICTION, a program proprietary to Genetics Institute, operates according to a different heuristic. The results of all three programs were used to create a prediction matrix across the segment of genomic DNA.

The three gene finding programs yielded a range of results. GRAIL identified the greatest percentage of genomic sequence as putative coding region, 2% of the data analyzed. GENEFINDER was second, calling 1%, and DICTION yielded the least putative coding region, with 0.8% of genomic sequence called as coding region.

The consensus data were as follows. GRAIL and GENEFINDER agreed on 0.7% of genomic sequence, GRAIL and DICTION agreed on 0.5% of genomic sequence, and the three programs together agreed on 0.25% of the data analyzed. That is, 0.25% of the genomic sequence was identified by all three of the programs as containing putative coding region.

Exons predicted by any two of the three programs ("consensus exons") were assorted into "gene bins" using two criteria: (1) any 7 consecutive exons within a 25 kb window were placed together in a bin as likely contributing to a single gene, and (2) all exons within a 25 kb window were placed together in a bin as likely contributing to a single gene if fewer than 7 exons were found within the 25 kb window.

PCR

The largest exon from each gene bin that did not span repetitive sequence was then chosen for amplification, as were all consensus exons longer than 500 bp. This method approximated one exon per gene; however, a number of genes were found to be represented by multiple elements.

Previously, we had determined that DNA fragments fewer than 250 bp in length do not bind well to the amino-modified glass surface of the slides used as support substrate for construction of microarrays; therefore, amplicons were designed in the present experiments to approximate 500 bp in length.

Accordingly, after selecting the largest exon per gene bin, a 500 bp fragment of sequence centered on the exon was passed to the primer picking software, PRIMER3 (available online for use at http://www-genome.wi.mit.edu/cgi-bin/primer/ ). A first additional sequence was commonly added to each exon-unique 5' primer, and a second, different, additional sequence was commonly added to each exon-unique 3' primer, to permit subsequent reamplification of the amplicon using a single set of "universal" 5' and 3' primers, thus immortalizing the amplicon. The addition of universal priming sequences also facilitates sequence verification, and can be used to add a cloning site should some exons be found to warrant further study.

The exons were then PCR amplified from genomic DNA, verified on agarose gels, and sequenced using the universal primers to validate the identity of the amplicon to be spotted in the microarray.

Primers were supplied by Operon Technologies (Alameda, CA). PCR amplification was performed by standard techniques using human genomic DNA (Clontech, Palo Alto, CA) as template. Each PCR product was verified by SYBR^{®} green (Molecular Probes, Inc., Eugene, OR) staining of agarose gels, with subsequent imaging by Fluorimager (Molecular Dynamics, Inc., Sunnyvale, CA). PCR amplification was classified as successful if a single band appeared.

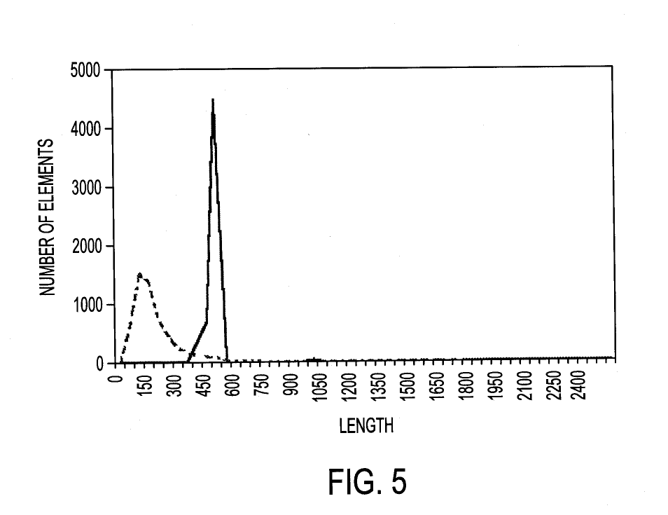

The success rate for amplifying exons of interest directly from genomic DNA using PCR was approximately 75%. FIG. 5 graphs the distribution of predicted exon length and distribution of amplified PCR products, with exon length shown by dashed line and PCR product length shown by solid line. Although the range of exon sizes is readily seen to extend to beyond 900 bp, the mean predicted exon size was only 229 bp, with a median size of 150 bp (n=9498). With an average amplicon size of 475± 25 bp, approximately 50% of the average PCR amplification product contained predicted coding region, with the remaining 50% of the amplicon containing either intron, intergenic sequence, or both.

Using a strategy predicated on amplifying about 500 bp, it was found that long exons had a higher PCR failure rate. To address this, the bioinformatics process was adjusted to amplify 1000, 1500 or 2000 bp fragments from exons larger than 500 bp. This improved the rate of successful amplification of exons exceeding 500 bp, constituting about 9.2% of the exons predicted by the gene finding algorithms.

Approximately 75% of the probes disposed on the array (90% of those that successfully PCR amplified) were sequence-verified by sequencing in both the forward and reverse direction using MegaBACE sequencer (Molecular Dynamics, Inc., Sunnyvale, CA), universal primers, and standard protocols.

Some genomic clones (BACs) yielded very poor PCR and sequencing results. The reasons for this are unclear, but may be related to the quality of early draft sequence or the inclusion of vector and host contamination in some submitted sequence data.

Although the intronic and intergenic material flanking coding regions could theoretically interfere with hybridization during microarray experiments, subsequent empirical results demonstrated that differential expression ratios were not significantly affected by the presence of noncoding sequence. The variation in exon size was similarly found not to affect differential expression ratios significantly; however, variation in exon size was observed to affect the absolute signal intensity (data not shown).

The 350 MB of genomic DNA was, by the above-described process, reduced to 9750 discrete probes, which were spotted in duplicate onto glass slides using commercially available instrumentation (MicroArray GenII Spotter and/or MicroArray GenIII Spotter, Molecular Dynamics, Inc., Sunnyvale, CA). Each slide additionally included either 16 or 32 E. coli genes, the average hybridization signal of which was used as a measure of background biological noise.

Each of the probe sequences was BLASTed against the human EST data set, the NR data set, and SwissProt GenBank (May 7, 1999 release 2.0.9).

One third of the probe sequences (as amplified) produced an exact match (BLAST Expect ("E") values less than 1e-100 (1 x 10⁻¹⁰⁰)) to either an EST (20% of sequences) or a known mRNA (13% of sequences). A further 22% of the probe sequences showed some homology to a known EST or mRNA (BLAST E values from 1e-5 (1 x 10⁻⁵) to 1e-99 (1 x 10⁻⁹⁹)). The remaining 45% of the probe sequences showed no significant sequence homology to any expressed, or potentially expressed, sequences present in public databases.

All of the probe sequences (as amplified) were then analyzed for protein similarities with the SwissProt database using BLASTX, Gish et al., Nature Genet. 3:266 (1993). The predicted functional breakdowns of the 2/3 of probes identical or homologous to known sequences are presented in Table 1.

| Total | V6 chip | V7 chip | Function Predicted from Comparative Sequence Analysis |
|---|---|---|---|
| 211 | 96 | 115 | Receptor |
| 120 | 43 | 77 | Zinc Finger |
| 30 | 11 | 19 | Homeobox |
| 25 | 9 | 16 | Transcription Factor |
| 17 | 11 | 7 | Transcription |
| 118 | 57 | 61 | Structural |
| 95 | 39 | 56 | Kinase |
| 36 | 18 | 18 | Phosphatase |
| 83 | 31 | 52 | Ribosomal |
| 45 | 19 | 26 | Transport |
| 21 | 7 | 14 | Growth Factor |
| 17 | 12 | 5 | Cytochrome |
| 50 | 33 | 17 | Channel |

As can be seen, the two most common types of genes were transcription factors and receptors, making up 2.2% and 1.8% of the arrayed elements, respectively.

EXAMPLE 2

Gene Expression Measurements From Genome-Derived Single Exon Microarrays

The two genome-derived single exon microarrays prepared according to Example 1 were hybridized in a series of simultaneous two-color fluorescence experiments to (1) Cy3-labeled cDNA synthesized from message drawn individually from each of brain, heart, liver, fetal liver, placenta, lung, bone marrow, HeLa, BT 474, or HBL 100 cells, and (2) Cy5-labeled cDNA prepared from message pooled from all ten tissues and cell types, as a control in each of the measurements. Hybridization and scanning were carried out using standard protocols and Molecular Dynamics equipment.

Briefly, mRNA samples were bought from commercial sources (Clontech, Palo Alto, CA and Amersham Pharmacia Biotech (APB)). Cy3-dCTP and Cy5-dCTP (both from APB) were incorporated during separate reverse transcriptions of 1 μg of polyA⁺ mRNA performed using 1 μg oligo(dT)12-18 primer and 2 μg random 9mer primers as follows. After heating to 70ºC, the RNA:primer mixture was snap cooled on ice. After snap cooling on ice, added to the RNA to the stated final concentration was: 1X Superscript II buffer, 0.01 M DTT, 100μM dATP, 100 μM dGTP, 100 μM dTTP, 50 μM dCTP, 50 μM Cy3-dCTP or Cy5-dCTP 50 μM, and 200 U Superscript II enzyme. The reaction was incubated for 2 hours at 42ºC. After 2 hours, the first strand cDNA was isolated by adding 1 U Ribonuclease H, and incubating for 30 minutes at 37ºC. The reaction was then purified using a Qiagen PCR cleanup column, increasing the number of ethanol washes to 5. Probe was eluted using 10 mM Tris pH 8.5.

Using a spectrophotometer, probes were measured for dye incorporation. Volumes of both Cy3 and Cy5 cDNA corresponding to 50 pmoles of each dye were then dried in a Speedvac, resuspended in 30 μl hybridization solution containing 50% formamide, 5X SSC, 0.2 μg/μl poly(dA), 0.2 μg/μl human cₒt1 DNA, and 0.5 % SDS.

Hybridizations were carried out under a coverslip, with the array placed in a humid oven at 42ºC overnight. Before scanning, slides were washed in 1X SSC, 0.2% SDS at 55ºC for 5 minutes, followed by 0.1X SSC, 0.2% SDS, at 55ºC for 20 minutes. Slides were briefly dipped in water and dried thoroughly under a gentle stream of nitrogen.

Slides were scanned using a Molecular Dynamics Gen3 scanner, as described. Schena (ed.), Microarray Biochip: Tools and Technology, Eaton Publishing Company/BioTechniques Books Division (2000) (ISBN: 1881299376).

Although the use of pooled cDNA as a reference permitted the survey of a large number of tissues, it attenuates the measurement of relative gene expression, since every highly expressed gene in the tissue/cell type-specific fluorescence channel will be present to a level of at least 10% in the control channel. Because of this fact, both signal and expression ratios (the latter hereinafter, "expression" or "relative expression") for each probe were normalized using the average ratio or average signal, respectively, as measured across the whole slide.

Data were accepted for further analysis only when signal was at least three times greater than biological noise, the latter defined by the average signal produced by the E. coli control genes.

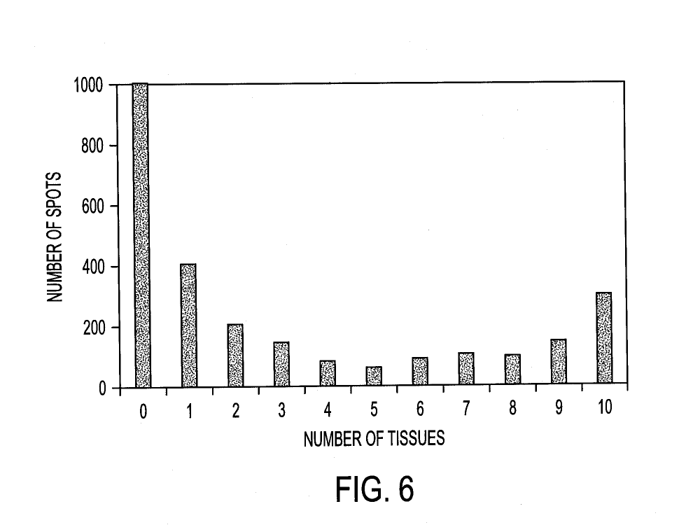

The relative expression signal for these probes was then plotted as a function of tissue or cell type, and is presented in FIG. 6.

FIG. 6 shows the distribution of expression across a panel of ten tissues. The graph shows the number of sequence-verified products that were either not expressed ("0"), expressed in one or more but not all tested tissues ("1" - "9"), and expressed in all tissues tested ("10").

Of 9999 arrayed elements on the two microarrays (including positive and negative controls and "failed" products), 2353 (51%) were expressed in at least one tissue or cell type. Of the gene elements showing significant signal—where expression was scored as "significant" if the normalized Cy3 signal was greater than 1, representing signal 5-fold over biological noise (0.2) — 39% (991) were expressed in all 10 tissues. The next most common class (15%) consisted of gene elements expressed in only a single tissue.

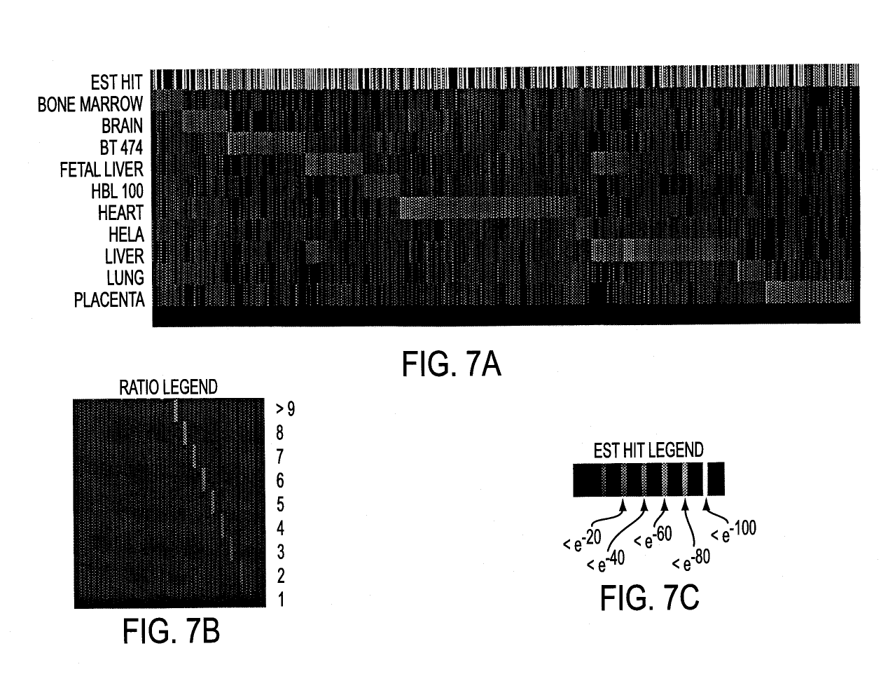

The genes expressed in a single tissue were further analyzed, and the results of the analyses are compiled in FIG. 7.

FIG. 7A is a matrix presenting the expression of all verified sequences that showed signal intensity greater than 3 in at least one tissue. Each clone is represented by a column in the matrix. Each of the 10 tissues assayed is represented by a separate row in the matrix, and relative expression (expression ratio) of a clone in that tissue is indicated at the respective node by intensity of green shading, with the intensity legend shown in panel B. The top row of the matrix ("EST Hit") contains "bioinformatic" rather than "physical" expression data—that is, presents the results returned by query of EST, NR and SwissProt databases using the probe sequence. The legend for "bioinformatic expression" (i.e., degree of homology returned) is presented in panel C. Briefly, white is known, black is novel, with gray depicting nonidentical with significant homology (white: E values < 1e-100 (1x10⁻¹⁰⁰); gray: E values from 1e-5 (1 x 10⁻⁵) to 1e-99 (1 x 10⁻⁹⁹); black: E values > 1e-5 (1 x 10⁻⁵)).

As FIG. 7 readily shows, heart and brain were demonstrated to have the greatest numbers of genes that were shown to be uniquely expressed in the respective tissue. In brain, 200 uniquely expressed genes were identified; in heart, 150. The remaining tissues gave the following figures for uniquely expressed genes: liver, 100; lung, 70; fetal liver, 150; bone marrow, 75; placenta, 100; HeLa, 50; HBL, 100; and BT474, 50.

It was further observed that there were many more "novel" genes among those that were up-regulated in only one tissue, as compared with those that were down-regulated in only one tissue. In fact, it was found that exons whose expression was measurable in only a single of the tested tissues were represented in sequencing databases at a rate of only 11%, whereas 36% of the exons whose expression was measurable in 9 of the tissues were present in public databases. As for those exons expressed in all ten tissues, fully 45% were present in existing expressed sequence databases. These results are not unexpected, since genes expressed in a greater number of tissues have a higher likelihood of being, and thus of having been, discovered by EST approaches.

Comparison of Signal from Known and Unknown Genes

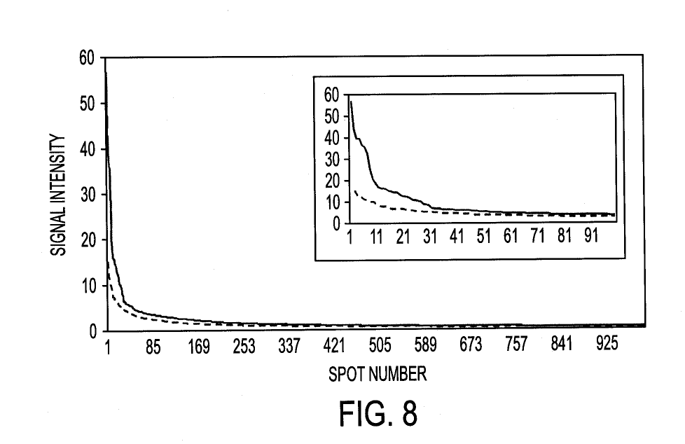

The normalized signal of the genes found to have high homology to genes present in the GenBank human EST database were compared to the normalized signal of those genes not found in the GenBank human EST database. The data are shown in FIG. 8.

FIG. 8 shows in dashed line the normalized Cy3 signal intensity for all sequence-verified products with a BLAST Expect ("E") value of greater than 1e-30 (1x10⁻³⁰) (designated "unknown") upon query of existing EST, NR and SwissProt databases, and shows in solid line the normalized Cy3 signal intensity for all sequence-verified products with a BLAST Expect value of less than 1e-30 (1 x 10⁻³⁰) ("known"). Note that biological background noise has an averaged normalized Cy3 signal intensity of 0.2.

As expected, the most highly expressed of the exons were "known" genes. This is not surprising, since very high signal intensity correlates with very commonly-expressed genes, which have a higher likelihood of being found by EST sequence.

However, a significant point is that a large number of even the high expressers were "unknown". Since the genomic approach used to identify genes and to confirm their expression does not bias exons toward either the 3' or 5' end of a gene, many of these high expression genes will not have been detected in an end-sequenced cDNA library.

The significant point is that presence of the gene in an EST database is not a prerequisite for incorporation into a genome-derived microarray, and further, that arraying such "unknown" exons can help to assign function to as-yet undiscovered genes.

Verification of Gene Expression

To ascertain the validity of the approach described above to identify genes from raw genomic sequence, expression of two of the probes was assayed using reverse transcriptase polymerase chain reaction (RT PCR) and northern blot analysis.

Two microarray probes were selected on the basis of exon size, prior sequencing success, and tissue-specific gene expression patterns as measured by the microarray experiments. The primers originally used to amplify the two respective exons from genomic DNA were used in RT PCR against a panel of tissue-specific cDNAs (Rapid-Scan gene expression panel 24 human cDNAs) (OriGene Technologies, Inc., Rockville, MD).

Sequence AL079300-1 was shown by microarray hybridization to be present in cardiac tissue, and sequence AL031734-1 was shown by microarray experiment to be present in placental tissue (data not shown). RT-PCR on these two sequences confirmed the tissue-specific gene expression as measured by microarrays, as ascertained by the presence of a correctly sized PCR product from the respective tissue type cDNAs.

Clearly, all microarray results cannot, and indeed should not, be confirmed by independent assay methods, or the high throughput, highly parallel advantages of microarray hybridization assays will be lost. However, in addition to the two RT-PCR results presented above, the observation that 1/3 of the arrayed genes exist in expression databases provides powerful confirmation of the power of our methodology — which combines bioinformatic prediction with expression confirmation using genome-derived single exon microarrays — to identify novel genes from raw genomic data.

To verify that the approach further provides correct characterization of the expression patterns of the identified genes, a detailed analysis was performed of the microarrayed sequences that showed high signal in brain.

For this latter analysis, sequences that showed high (normalized) signal in brain, but which showed very low (normalized) signal (less than 0.5, determined to be biological noise) in all other tissues, were further studied. There were 82 sequences that fit these criteria, approximately 2% of the arrayed elements. The 10 sequences showing the highest signal in brain in microarray hybridizations are detailed in Table 2, along with assigned function, if known or reasonably predicted.

| Microarray Sequence Name | Normalized Signal | Expression Ratio | Homology to EST present in GenBank | Gene Function as described by GenBank |
|---|---|---|---|---|
| AP000217-1 | 5.2 | + 7.7 | High | S-100 protein, b-chain, Ca²⁺ binding protein expressed in central nervous system |
| AP000047-1 | 2.3 | | High | Unknown Function |
| AC006548-9 | 1.7 | | High | Similar to mouse membrane glyco-protein M6, expressed in central nervous system |
| AC007245-5 | 1.5 | | High | Similar to amphiphysin, a synaptic vesicle-associated protein. Ref 21 |
| L44140-4 | 1.2 | + 2.0 | High | Endothelial actin-binding protein found in nonmuscle filamin |
| AC004689-9 | 1.2 | + 3.5 | High | Protein Phosphatase PP2A, neuronal/downregulates activated protein kinases |
| AL031657-1 | 1.2 | + 3.0 | High | Unknown function/ Contains the anhyrin motif, a common protein sequence motif |
| AC009266-2 | 1.1 | + 3.7 | Low | Low homology to the Synaptotagmin I protein in rat/present at low levels throughout rat brain |
| AP000086-1 | 1.0 | + 2.7 | Low | Unknown, very poor homology to collagen |
| AC004689-3 | 1.0 | | High | Protein Phosphatase PP2A, neuronal/downregulates activated protein kinases |

Of the ten sequences studied by these latter confirmatory approaches, eight were previously known. Of these eight, six had previously been reported to be important in the central nervous system or brain. The exon giving the highest signal (AP00217-1) was found to be the gene encoding an S100B Ca²⁺ binding protein, reported in the literature to be highly and uniquely expressed in the central nervous system. Heizmann, Neurochem. Res. 9:1097 (1997).

A number of the brain-specific probe sequences (including AC006548-9, AC009266-2) did not have homology to any known human cDNAs in GenBank but did show homology to rat and mouse cDNAs. Sequences AC004689-9 and AC004689-3 were both found to be phosphatases present in neurons (Millward et al., Trends Biochem. Sci. 24(5):186-191 (1999)). Two microarray sequences, AP000047-1 and AP000086-1 have unknown function, with AP000086-1 being absent from GenBank. Functionality can now be narrowed down to a role in the central nervous system for both of these genes, showing the power of designing microarrays in this fashion.

Next, the function of the chip sequences with the highest (normalized) signal intensity in brain, regardless of expression in other tissues, was assessed. In this latter analysis, we found expression of many more common genes, since the sequences were not limited to those expressed only in brain. For example, looking at the 20 highest signal intensity spots in brain, 4 were similar to tubulin (AC00807905; AF146191-2; AC007664-4; AF14191-2), 2 were similar to actin (AL035701-2; AL034402-1), and 6 were found to be homologous to glyceraldehyde-3-phosphate dehydrogenase (GAPDH) (AL035604-1; Z86090-1; AC006064-L, AC006064-K; AC035604-3; AC006064-L). These genes are often used as controls or housekeeping genes in microarray experiments of all types.

Other interesting genes highly expressed in brain were a ferritin heavy chain protein, which is reported in the literature to be found in brain and liver (Joshi et al., J. Neurol. Sci. 134(Suppl):52-56 (1995)), a result confirmed with the array. Other highly expressed chip sequences included a translation elongation factor 1α (AC007564-4), a DEAD-box homolog (AL023804-4), and a Y-chromosome RNA-binding motif (Chai et al., Genomics 49(2):283-89 (1998))(AC007320-3). A low homology analog (AP00123-1/2) to a gene, DSCR1, thought to be involved in trisomy 21 (Down's syndrome), showed high expression in both brain and heart, in agreement with the literature (Fuentes et al., Mol. Genet. 4(10):1935-44 (1995)).

As a further validation of the approach, we selected the BAC AC006064 to be included on the array. This BAC was known to contain the GAPDH gene, and thus could be used as a control for the exon selection process. The gene finding and exon selection algorithms resulted in choosing 25 exons from BAC AC006064 for spotting onto the array, of which four were drawn from the GAPDH gene. Table 3 shows the comparison of the average expression ratio for the 4 exons from BAC006064 compared with the average expression ratio for 5 different dilutions of a commercially available GAPDH cDNA (Clontech).

| | AC006064 (n = 4) | Control ( n = 5) |
|---|---|---|
| Bone Marrow | -1.81 ±0.11 | -1.85 ±0.08 |
| Brain | -1.41 ±0.11 | -1.17 ±0.05 |
| BT474 | 1.85 ±0.09 | 1.66 ±0.12 |
| Fetal Liver | -1.62 ±0.07 | -1.41 ±0.05 |
| HBL100 | 1.32 ±0.05 | 2.64 ±0.12 |
| Heart | 1.16 ±0.09 | 1.56 ±0.10 |
| HeLa | 1.11 ±0.06 | 1.30 ±0.15 |
| Liver | -1.62 ±0.22 | -2.07 ± |
| Lung | -4.95 ±0.93 | -3.75 ±0.21 |
| Placenta | -3.56 ±0.25 | -3.52 ±0.43 |

Each tissue shows excellent agreement between the experimentally chosen exons and the control, again demonstrating the validity of the present exon mining approach. In addition, the data also show the variability of expression of GAPDH within tissues, calling into question its classification as a housekeeping gene and utility as a housekeeping control in microarray experiments.

EXAMPLE 3

Representation of Sequence and Expression Data as a "Mondrian"

For each genomic clone processed for microarray as above-described, a plethora of information was accumulated, including full clone sequence, probe sequence within the clone, results of each of the three gene finding programs, EST information associated with the probe sequences, and microarray signal and expression for multiple tissues, challenging our ability to display the information.

Accordingly, we devised a new tool for visual display of the sequence with its attendant annotation which, in deference to its visual similarity to the paintings of Piet Mondrian, is hereinafter termed a "Mondrian". FIGS. 3 and 4 present the key to the information presented on a Mondrian.

FIG. 9 presents a Mondrian of BAC AC008172 (bases 25,000 to 130,000 shown), containing the carbamyl phosphate synthetase gene (AF154830.1). Purple background within the region shown as field 81 in FIG. 3 indicates all 37 known exons for this gene.

As can be seen, GRAIL II successfully identified 27 of the known exons (73%), GENEFINDER successfully identified 37 of the known exons (100%), while DICTION identified 7 of the known exons (19%).

Seven of the predicted exons were selected for physical assay, of which 5 successfully amplified by PCR and were sequenced. These five exons were all found to be from the same gene, the carbamyl phosphate synthetase gene (AF154830.1).

The five exons were arrayed, and gene expression measured across 10 tissues. As is readily seen in the Mondrian, the five chip sequences on the array show identical expression patterns, elegantly demonstrating the reproducibility of the system.

FIG. 10 is a Mondrian of BAC AL049839. We selected 12 exons from this BAC, of which 10 successfully sequenced, which were found to form between 5 and 6 genes. Interestingly, 4 of the genes on this BAC are protease inhibitors. Again, these data elegantly show that exons selected from the same gene show the same expression patterns, depicted below the red line. From this figure, it is clear that our ability to find known genes is very good. A novel gene is also found from 86.6 kb to 88.6 kb, upon which all the exon finding programs agree. We are confident we have two exons from a single gene since they show the same expression patterns and the exons are proximal to each other. Backgrounds in the following colors indicate a known gene (top to bottom): red=kallistatin protease inhibitor (P29622); purple= plasma serine protease inhibitor (P05154); turquoise = α1 anti-chymotrypsin (P01011); mauve = 40S ribosomal protein (P08865). Note that chip sequence 8 and 12 did not sequence verify.

EXAMPLE 4

Sequences of Genes Identified From Genomic Sequence By Gene Prediction and Single Exon Microarray Analysis

The sequences of three exons identified from human genomic sequence in experiments as set forth in Examples 1 - 3 are presented here, with each exon represented by its predicted coding sequence, and thereafter by the sequence of the amplicon as used on the genome-derived single exon microarray to assess its expression. The three sequences were chosen, respectively, to represent each of three classes of genes obtainable by this method: (1) those that have already been identified and accessioned into expression databases such as EST, SNP, SwissProt databases; (2) those that are not identically represented in expression databases, but that have sequence showing significant homology to genes already present in such expression databases; and (3) those that are neither identically present nor have significant sequence homology to genes present in expression databases.

The first, designated AC007683-4-chip.seq.1, was found to be identical to a sequence in an existing expression database.

AC007683-4-chip.seq.1 predicted exon:

TTTTTTTTTTTGCAAGCAGATAAAGGCTTATTTTACTTTAATGGCTGATCTATGTA ATCACGGAGGCCAGTATGTACACACAAAGGGGCAGCTTTTATTTCTTGGTCTCTT CCTCCTTGGACAAAGTCTTGATGATCTCCTCCTTCTTGGCCTGGAGGTGCTCTTC ATAGCTCTTGTGTGCTTCCTTGGTCTTAGATCTGCGGGCCTCAGCCTGATCAGCC AGGAGCTTCTTGCGGGCCTTGTCTGCCTTCAGCTTGTGGATGTGTTCCATGAGAA TCTGCTTGTTTTTTAACACATTCCTCTTCACCTTCAGGTACAGGCTGTGATACATG CGGCGATCAATCTTCTTA [SEQ ID NO:1]

AC007683-4-chip.seq.1 amplicon:

CAGTCCACATGGGTACAAGCCCTGAAACCTCAAATGTACATCAGAATTACCTGTG GAGTTGTTTTTTTTTTTTTTTTTTTTTTTTTGCAAGCAGATAAAGGCTTATTTTACT TTAATGGCTGATCTATGTAATCACGGAGGCCAGTATGTACACACAAAGGGGCAGC TTTTATTTCTTGGTCTCTTCCTCCTTGGACAAAGTCTTGATGATCTCCTCCTTCTT GGCCTGGAGGTGCTCTTCATAGCTCTTGTGTGCTTCCTTGGTCTTAGATCTGCGG GCCTCAGCCTGATCAGCCAGGAGCTTCTTGCGGGCCTTGTCTGCCTTCAGCTTGT GGATGTGTTCCATGAGAATCTGCTTGTTTTTTAACACATTCCTCTTCACCTTCAGG TACAGGCTGTGATACATGCGGCGATCAATCTTCTTAGATTCACGGTATCTTCTGA GCAGCCGGTGCAGAATCCTCATTCTCCTCATCCACGTGACCTTCTCTGGCATTCG G [SEQ ID NO:2].

The second, designated AC007682-2-chip.seq.2, was not found identically in an expression database, but was found to have homology to one or more sequences in such databases.

AC007682-2-chip.seq.2 predicted exon:

TATGGTATTTTCTTATAGCAACAAAAAATAAAGATGGGGTGGAGAAATATA TTTATAGAAAGTATTTTTTTAAGT [SEQ ID NO:3]

AC007682-2-chip.seq.2 amplicon:

AGTATGGAGCCCCCTTCATGGGACAGGTGGCTTTAAGAAGAGGAAGAGAGACCT GAGCTGGCAGGGACTCTCTTACCCTCTCACCATGTGATGCCCTCCACATGTTATG ATGCAGCAAGAAGGCCCTCACTGGTTGCTAGTGCCATGCTCTTCGACTTCCCAGC CTGCAGAACTATAAGAAATAAACTTATTTTCTTTATAACTTACACATTTATGGTAT TTTCTTATAGCAACAAAAAATAAAGATGGGGTGGAGAAATATATTTATAGAAAGT ATTTTTTTAAGTAAATGAGAAATTAGACATAATGTTTTTAACTCTAGAGAAATTGA AAACAGAGCACAGCACATCGGATAAATTCAATAACTATCTTAAGAATCAGCAAAA CAACATGCAGATGGCTGATTGGCAATAGTTTCAGTAGGCAGATTTTGATTAAAAT AAAGAAAAACTTTTTAATAATTAAACCTCTCCTTAAAACATTATGACTTTATGAGG TAA [SEQ ID NO:4]

The third exon, designated AC007552-4-chip.seq.2, was neither identically present nor significantly related in sequence to any entry in a public expression database.

AC007552-4-chip.seq.2 Predicted Exon:

TCTTCATTATTAATCACTCTTAAACCTCTTCTTCAATCTTCTCCTCATGTTTAAT TTCTCCCTTATCTTATCTTCATAACTCAGTGCCATTCTCCCTTCATAACAACAGAAGC TGACATTGGAGG [SEQ ID NO:5]

AC007552-4-chip.seq.2 amplicon:

TCATCCTAATTTATATAAAGCACACTACAATCTTAATTTAACAATCCATTCCA AATTCCAATAATCTCCAGTGTTGAGATATTTTTTCCATACAGCCTAAAGTGCACAT ATTTAGACATTTCTCCACCCATCTCCTTTGCACACGAAAAGTTGGTAAACGACCTC ATTATACTAGTAGCCTTTCATATTCTTCATTATTAATCACTCTTAAACCTCTTCTTC AATCTTCTCCTCATGTTTAATTTCTCCCTTATCTTATCTTCATAACTCAGTGCCATT CTCCCTTCATAACAACAGAAGCTGACATTGGAGGAGTATCAGCCAATGTGTACCG CTCTTTCCCTACTGTGGTCCACTGTCACCCCTAACTATTTTATGAATAGGATTCCT ATTTCTAGAGAAGAAAACGCAGACTTGGAGAGGTTGAGTAAGTTGCCTAGGAATG TGAAGCTGGGGTGTAGCAGAAGGGGGTCGACGTCAGGTCTGGATACCTCACCGT G [SEQ ID NO:6]

EXAMPLE 5

Genome-Derived Single Exon Probes Useful For Measuring Human Gene Expression

The protocols set forth in Examples 1 and 2, supra, were applied with some modification to additional human genomic sequence as it became newly available in GenBank. From the collective efforts of these and the experiments reported in Example 2, we generated over 15,000 unique human genome-derived single exon probes that could be shown to be expressed at significant levels in one or more of ten tested tissues.

Modifications to the protocols for bioinformatic prediction of exons set forth in Examples 1 and 2 were as follows.

First, we added a fourth gene prediction program, GENSCAN, to the three originally used, DICTION, GENEFINDER, and GRAIL.

Second, we increased the resolution of our exon predictions, as follows.

In the experiments reported in Examples 1 and 2, we applied a 25 bp window in scanning genomic sequence: exons were called when any two of the three gene prediction programs identified an exon anywhere within the window. In the more recent experiments, we looked for consensus on a nucleotide by nucleotide basis: when any two or more of the four programs identified the nucleotide as falling within an exon, the nucleotide was called as belonging to an exon. This had the additional benefit of merging overlapping predicted exons.

Finally, we applied a lower size threshold of 75 contiguous nucleotides to each consensus exon.

Each probe was completely sequenced on both strands prior to its use on a genome-derived single exon microarray; sequencing confirmed the exact chemical structure of each probe. An added benefit of sequencing is that it placed us in possession of a set of single base-incremented fragments of the sequenced nucleic acid, starting from the sequencing primer 3' OH. (Since the single exon probes were first obtained by PCR amplification from genomic DNA, we were of course additionally in possession of an even larger set of single base incremented fragments of each of the single exon probes, each fragment corresponding to an extension product from one of the two amplification primers.) Hybridization analysis was conducted essentially as set forth in Examples 1 and 2, with one modification.

In Examples 1 and 2, we used a pool of 10 tissues/cell types as control. We have since observed that every probe that demonstrates expression in the control pool can readily be shown to be expressed in HeLa cells, and have used HeLa as the source of control message in the more recent experiments.

In the analysis of hybridization results, the uniform absolute signal intensity threshold used in Examples 1 and 2 to identify signals large enough to be considered biologically significant (0.5, representing a level roughly 10 times greater than the average of all E. coli control spots on a first iteration chip) was replaced with a statistical threshold determined for each channel and each hybridization as follows.

Starting typically with 32 E. coli sequences, spotted in duplicate (left and right side) for a total of 64 control spots per microarray, control spots were eliminated if we observed more than a five-fold difference between the left and right side raw (unnormalized) signals for the probe.

The median of the normalized signal from the remaining control spots was calculated (see infra for normalization routine).

Control spots were eliminated as outliers if they had signal intensity greater than the median of the normalized signals plus 2.4 (where 2.4 is roughly 12 times the observed standard deviation of control spot populations) and normalization was performed as set forth below.

The mean and standard deviation of the normalized signal intensity from the remaining control spots were calculated, and the mean plus three standard deviations of the controls was then applied as a minimum intensity threshold for the particular hybridization experiment, giving a 99% confidence that expression is significant.

Signal normalization was accomplished as follows. For each hybridization (each microarray, separately for each of the two colors), the median value of all of the spots was determined. For each probe, the normalized signal value is the arithmetic mean of the probe's duplicate intensities (each DNA probe, including controls, is spotted twice per slide) divided by the population median.

Using this threshold, we identified over 15,000 single exon probes that produce significant signal in one or more of ten tested tissues/cell types. The exact structures of these single exon probes are clearly presented in the SEQUENCE LISTINGs included in commonly owned and copending U.S. provisional application nos. 60/207,456, filed May 26, 2000; 60/234,687, filed September 21, 2000; 60/236,359, filed September 27, 2000; in commonly owned and copending U.K. patent application no. 0024263.6, filed October 4, 2000; and in commonly owned and copending PCT applications PCT/US01/00666; PCT/US01/00667; PCT/US01/00664; PCT/US01/00669; PCT/US01/00665; PCT/US01/00668; PCT/US01/00663; PCT/US01/00662; PCT/US01/00661; and PCT/US01/00670, the disclosures of which are incorporated herein by reference in their entireties.

We also predicted the sequence of the ORF within the exon of each of the probes, where ORF was defined as that portion of an exon that can be translated in its entirety into a sequence of contiguous amino acids.

To predict the ORF, we first looked for consensus as between any two or more of the four gene prediction programs. Consensus was required in two parameters: (1) as with prediction of the exon, each nucleotide must have been identified by two or more programs as falling within an exon; and, additionally, (2) the programs relied upon to establish that consensus must have agreed on the frame. Presence of a stop codon disqualified the predicted ORF. ORFs shorter than 50 nt were also disregarded.

Absent consensus as to nucleotide and frame, each of the six frames of the predicted exon were examined individually for stop codons and the longest open reading frame of at least 51 nt selected as the exon's likely ORF. Certain of the exons have no ORF as defined by either set of criteria.

We then translated the predicted ORFs using the standard genetic code.

The exact structures of these single exon probes are clearly presented in the SEQUENCE LISTINGs included in commonly owned and copending U.S. provisional application nos. 60/207,456 filed May 26, 2000; 60/234,687, filed September 21, 2000; 60/236,359, filed September 27, 2000; in commonly owned and copending U.K. patent application no. 0024263.6, filed October 4, 2000; and in commonly owned and copending PCT applications PCT/US01/00666; PCT/US01/00667; PCT/US01/00664; PCT/US01/00669; PCT/US01/00665; PCT/US01/00668; PCT/US01/00663; PCT/US01/00662; PCT/US01/00661; and PCT/US01/00670, the disclosures of which are incorporated herein by reference in their entireties.

The sequence of each of the probes, exons, and ORF-encoded peptides was used as a query to identify the most similar sequence in each of dbEST, GenBank NR, and SWISSPROT. The query programs used were BLAST (nucleic acid sequence query of dbEST and NR), BLASTX (nucleic acid sequence query of SWISSPROT), TBLASTX (peptide sequence query of dbEST and NR), and BLASTP (peptide sequence query of SWISSPROT). Because the query sequences are themselves derived from genomic sequence in GenBank, only nongenomic hits from NR were scored.

The attached SEQUENCE LISTINGs in our commonly owned and copending applications report, for each SEQ ID NO:, the accession number of the entry from each of the three queried databases that gave the highest absolute expect ("E") value (the "top hit"), along with the "E" value itself. The SEQUENCE LISTING is incorporated herein by reference in its entirety.

All patents, patent publications, and other published references mentioned herein are hereby incorporated by reference in their entireties as if each had been individually and specifically incorporated by reference herein. While preferred illustrative embodiments of the present invention are described, it will be apparent to one skilled in the art that various changes and modifications may be made therein without departing from the invention, and it is intended in the appended claims to cover all such changes, modifications and equivalents that fall within the true spirit and scope of the invention.

What is Claimed Is:

1. A single exon nucleic acid microarray, comprising:
a plurality of nucleic acid probes addressably disposed upon a substrate,
wherein at least 50% of said nucleic acid probes include a fragment of no more than one exon of a eukaryotic genome, said fragment selectively hybridizable at high stringency to an expressed gene, wherein said plurality of nucleic acid probes averages at least 100 bp in length, and wherein said eukaryotic genome averages at least one intron per gene.

2. The microarray of claim 1, wherein at least 95% of said nucleic acid probes include a selectively hybridizable portion of no more than one exon of said eukaryotic genome.

3. The single exon nucleic acid microarray of claim 1, wherein at least 50% of said exon-including nucleic acid probes further comprise, contiguous to a first end of said fragment, a first intronic and/or intergenic sequence that is identically contiguous to said fragment in the genome.

4. The single exon nucleic acid microarray of claim 1, wherein at least 95% of said exon-including nucleic acid probes further comprise, contiguous to a first end of said fragment, a first intronic and/or intergenic sequence that is identically contiguous to said fragment in the genome.

5. The single exon nucleic acid microarray of claim 1, wherein at least 50% of said exon-including nucleic acid probes comprise, contiguous to a first end of said fragment, a first intronic and/or intergenic sequence that is identically contiguous to said fragment in the human genome, and further comprise, contiguous to a second end of said fragment, a second intronic and/or intergenic sequence that is identically contiguous to said fragment in the human genome.

6. The single exon nucleic acid microarray of claim 1, wherein at least 95% of said exon-including nucleic acid probes comprise, contiguous to a first end of said fragment, a first intronic and/or intergenic sequence that is identically contiguous to said fragment in the human genome, and further comprise, contiguous to a second end of said fragment, a second intronic and/or intergenic sequence that is identically contiguous to said fragment in the human genome.

7. The single exon nucleic acid microarray of claim 1, wherein at least 50% of said exon-including nucleic acid probes lack prokaryotic and bacteriophage vector sequence.

8. The single exon nucleic acid microarray of claim 1, wherein at least 95% of said exon-including nucleic acid probes lack prokaryotic and bacteriophage vector sequence.

9. The single exon nucleic acid microarray of claim 1, wherein at least 50% of said exon-including nucleic acid probes lack homopolymeric stretches of A or T.

10. The single exon nucleic acid microarray of claim 1, wherein at least 95% of said exon-including nucleic acid probes lack homopolymeric stretches of A or T.

11. The microarray of claim 1, wherein said eukaryotic genome averages at least two introns per gene.

12. The microarray of claim 1, wherein said eukaryotic genome averages at least three introns per gene.

13. The microarray of claim 1, wherein said eukaryotic genome averages at least five introns per gene.

14. The microarray of claim 1, wherein said genome is a human genome.

15. A method of identifying genes in a eukaryotic genome, comprising:
algorithmically predicting at least one of said gene's exons from genomic sequence of said eukaryote; and then
detecting hybridization of mRNA-derived nucleic acids to a nucleic acid probe having a selectively hybridizable portion identical in sequence to, or complementary in sequence to, said predicted exon,
wherein said probe is included within a single exon microarray according to any one of claims 1 - 14.

16. A method of measuring eukaryotic gene expression, comprising:
contacting the single exon microarray of any one of claims 1 - 14 with a first collection of detectably labeled nucleic acids, said first collection nucleic acids derived from mRNA of at least one eukaryotic tissue or cell type; and then
measuring the label detectably bound to each probe of said microarray.

17. The method of claim 16, further comprising comparing said measurement to a second measurement, said second measurement identically obtained using a second, control, collection of nucleic acids.

18. The method of claim 17, wherein said microarray is contacted simultaneously with said first and second collections of detectably labeled nucleic acids, wherein said first and second collection nucleic acids are distinguishably labeled.

19. A visual display of eukaryotic genomic sequence annotated with information about a predetermined biologic function, comprising:
a first visual element, each point along the length of which first visual element maps linearly and uniquely to a nucleotide of said genomic sequence;
a second visual element, first and second boundaries of which second visual element map linearly to a first and second nucleotide of said genomic sequence, wherein said first and second nucleotides delimit a region of said genomic sequence predicted to have said predetermined function; and
a third visual element, first and second boundaries of which third visual element map linearly to a first and second nucleotide of said genomic sequence, wherein said first and second nucleotides delimit a region of said genomic sequence experimentally confirmed to have said predetermined function.

20. The visual display of claim 19, wherein said display is electronic.

21. A high throughput, microarray-based method to confirm predicted exons, comprising:
detecting hybridization by transcript-derived nucleic acids to microarray probes that include genomic sequence predicted to contribute to no more than one exon,
detectable hybridization confirming the prediction of the exon included in each of said detectably hybridized probes.

22. The method of claim 21, wherein at least 75% of the probes of said microarray include genomic sequence predicted to contribute to no more than one exon.

23. The method of claim 21, wherein at least 90% of the probes of said microarray include genomic sequence predicted to contribute to no more than one exon

24. The method of claim 21, wherein at least 95% of the probes of said microarray include genomic sequence predicted to contribute to no more than one exon.

25. The method of claim 21, wherein said genomic sequence is human genomic sequence.

26. The method of claim 21, wherein said prediction is output from a computer program selected from the group consisting of GenScan, Diction, Genefinder, and Grail.

27. The method of claim 26, wherein said prediction is output from GenScan.

28. The method of claim 21, wherein said microarray has probes that collectively include exons predicted from all chromosomes of a eukaryotic organism.

29. The method of claim 28, wherein said eukaryotic organism is a human being.

30. The method of claim 21, wherein said microarray has probes that include exons predicted from human chromosome 22.

31. The method of claim 21, wherein each of said predicted exons is represented by a plurality of probes on said array.

32. The method of claim 21, wherein said microarray includes between 5,000 and 19,000 probes.

33. The method of claim 21, wherein the genomic sequence included within said probes is selected at least in part based upon considerations of base composition and/or hybridization binding stringency.

34. The method of claim 21, wherein said probes include at least 50 nt of predicted exon.

35. The method of claim 21, wherein said probes include at least 75 nt of predicted exon.

36. The method of claim 21, wherein said probes are amplified from genomic DNA.

37. The method of claim 21, wherein said probes are chemically synthesized.

38. The method of claim 21, wherein said probes are noncovalently attached to the substrate of said microarray.

39. The method of claim 21, wherein said probes are covalently attached to the substrate of said microarray.

40. The method of claim 21, wherein said probes are disposed on said microarray substrate by ink jet.

41. The method of claim 21, wherein the substrate of said microarray is a glass slide.

42. The method of claim 21, further comprising the antecedent step of:
contacting said microarray with at least a first sample of transcript-derived nucleic acids, said nucleic acids being detectably labeled.

43. The method of claim 42, wherein said transcript-derived nucleic acids are first strand cDNA.

44. The method of claim 43, wherein said cDNAs are fluorescently labeled.

45. The method of claim 44, wherein said fluorescent label is selected from the group consisting of Cy3 and Cy5.

46. The method of claim 42, wherein said contacting step comprises contacting said microarray concurrently with a first sample of transcript-derived nucleic acids and with a second sample of transcript-derived nucleic acids, wherein said first and second samples are labeled respectively with a first and a second label, said first and second labels being separately detectable.

47. The method of claim 46, wherein said detecting includes normalizing and background correcting signals from each of said labels.

48. The method of claim 46, wherein said labels are Cy3 and Cy5.

49. The method of claim 46, wherein said first sample includes transcript-derived nucleic acids pooled from a plurality of tissues and/or cell types.

50. The method of claim 49, wherein said pool includes transcript-derived nucleic acids from a plurality of human cell lines.

51. The method of claim 49, wherein the transcript-derived nucleic acids of said second sample are derived from a cell line or normal tissue.

52. The method of claim 51, wherein the transcript-derived nucleic acids of said second sample are derived from a source within the group of human tissues and cell lines consisting of: brain, heart, liver, fetal liver, placenta, lung, bone marrow, HeLa cells, BT474 cells and HBL 100 cells.

53. A method of identifying potential false positive exon predictions, comprising:
detecting hybridization by transcript-derived nucleic acids to a microarray that has probes that include genomic sequence predicted to contribute to no more than one exon,
absence of detectable hybridization identifying as a potential false positive the exon predicted in each undetectably hybridized probe.

54. A method of identifying one or more genes expressed by one or more eukaryotic cells having a genome that averages at least one intron per gene, comprising:
(a) contacting a cDNA sample prepared by enzymatically copying messenger RNA obtained from said eukaryotic cell(s) into cDNA, wherein said cDNA comprises a detectable label, with a plurality of single exon probes, each said single exon probe comprising a discrete nucleic acid sequence encoding all or a portion of a single exon of said eukaryotic genome that specifically hybridizes at high stringency to a target nucleic acid when said target nucleic acid is present in said cDNA sample;
(b) detecting a signal from each said single exon probe that is specifically hybridized to said target nucleic acid,
wherein the presence of said signal indicates the expression of a gene comprising said single exon by said eukaryotic cell(s).

55. A method of identifying one or more genes expressed by one or more human cells, comprising:
(a) contacting a cDNA sample prepared by copying messenger RNA obtained from said human cell(s) into cDNA using reverse transcriptase, wherein said cDNA comprises a detectable label, with a nucleic acid microarray, said microarray comprising a substantially planar glass substrate comprising (i) at least 5000 addressable locations to which single exon probes are bound, each said single exon probe comprising a discrete nucleic acid sequence encoding all or a portion of a single exon of a human genome that is specifically hybridizable at high stringency to a target nucleic acid, wherein said target nucleic acid is a sequence encoding all or a portion of an expressed gene, or a complementary sequence thereof, and (ii) one or more additional locations to which control nucleic acid sequences are bound; and
(b) generating a signal from each said addressable location,
wherein the presence of a signal at a specific addressable location indicates the expression by said human cell(s) of a gene comprising the single exon probe bound to that addressable location.

56. A high throughput, microarray-based method of grouping exons into a common gene, comprising:
comparing the patterns of tissue and/or cell-type expression of exons predicted from a contiguous region of genomic DNA,
wherein said patterns of expression have been determined by detecting hybridization of transcript-derived nucleic acids from a plurality of tissues and/or cell types to microarray probes, each of said probes including genomic sequence predicted to contribute to no more than one of said exons, said microarray including probes that collectively comprise all of said exons,
consensus in said expression patterns identifying exons that are groupable into a common gene.

57. The method of claim 56, wherein said gene is a human gene.

58. The method of claim 56, wherein said patterns are detected by detecting (i) fluorescence intensity, (ii) the ratio of intensity as between concurrently hybridized first and second samples, or (iii) a combination of (i) and (ii).

59. A nucleic acid microarray comprising:
a substrate comprising a plurality of addressable locations to which nucleic acid sequences are bound; and
a plurality of single exon probes bound at said addressable locations, each said single exon probe comprising a discrete nucleic acid sequence encoding all or a portion of a single exon of a eukaryotic genome averaging at least one intron per gene that is specifically hybridizable at high stringency to a target nucleic acid, wherein said target nucleic acid is a sequence encoding all or a portion of an expressed gene, or a complementary sequence thereof.

60. A nucleic acid microarray comprising:
a substantially planar glass substrate comprising (i) at least 5000 addressable locations to which single exon probes are bound, each said single exon probe comprising a discrete nucleic acid sequence encoding all or a portion of a single exon of a human genome that is specifically hybridizable at high stringency to a target nucleic acid, wherein said target nucleic acid is a sequence encoding all or a portion of an expressed gene, or a complementary sequence thereof, and (ii) one or more additional locations to which control nucleic acid sequences are bound.

61. A single exon nucleic acid microarray, comprising:
a plurality of nucleic acid probes addressably disposed upon a substrate,
wherein at least 50% of said probes include genomic sequence predicted to contribute to no more than one exon of a eukaryotic genome, said eukaryotic genome averaging at least one intron per gene, and wherein said plurality of nucleic acid probes averages at least 50 nt in length.

62. The microarray of claim 61, wherein at least 75% of said nucleic acid probes include genomic sequence predicted to contribute to no more than one exon of a eukaryotic genome.

63. The microarray of claim 61, wherein at least 90% of the probes of said microarray include genomic sequence predicted to contribute to no more than one exon of a eukaryotic genome.

64. The microarray of claim 61, wherein at least 95% of the probes of said microarray include genomic sequence predicted to contribute to no more than one exon of a eukaryotic genome.

65. The microarray of claim 61, wherein said microarray has probes that collectively include exons predicted from all chromosomes of a eukaryotic genome.

66. The microarray of claim 61, wherein said eukaryotic genome is a human genome.

67. The microarray of claim 65, wherein said eukaryotic genome is a human genome.

68. The microarray of claim 61, wherein said prediction is output from a computer program selected from the group consisting of GenScan, Diction, Genefinder, and Grail.

69. The microarray of claim 68, wherein said prediction is output from GenScan.

70. The microarray of claim 61, wherein each of said predicted exons is represented by a plurality of probes on said array.

71. The microarray of claim 61, wherein said microarray includes between 5,000 and 19,000 probes.

72. The microarray of claim 61, wherein the genomic sequence included within said probes is selected at least in part based upon considerations of base composition and/or hybridization binding stringency.

73. The microarray of claim 61, wherein said probes have been amplified from genomic DNA.

74. The microarray of claim 61, wherein said probes have been chemically synthesized.

75. The microarray of claim 61, wherein said probes are noncovalently attached to the substrate of said microarray.

76. The microarray of claim 61, wherein said probes are covalently attached to the substrate of said microarray.

77. The microarray of claim 61, wherein said probes are disposed on said microarray substrate by ink jet.

78. The microarray of claim 61, wherein said substrate is a glass slide.

79. The microarray of claim 61, wherein each of said probes is disposed on said array with its reverse complement.

80. The microarray of claim 61, further comprising control probes.

81. The microarray of claim 61, wherein at least 50% of said exon-including nucleic acid probes comprise, contiguous to a first end of said predicted exon, a first intronic and/or intergenic sequence that is identically contiguous to said exon in the human genome, and further comprise, contiguous to a second end of said predicted exon, a second intronic and/or intergenic sequence that is identically contiguous to said exon in the human genome.

82. A software data structure for annotating nucleic acid sequence with confirmed bioinformatic predictions, the data structure stored in a machine readable medium and comprising:
a plurality of sequence entries, each sequence entry including (i) a sequence identifier and (ii) software means for relating said sequence identifier to data that encode a confirmed prediction of a biological function of the nucleic acid sequence identified by said sequence identifier.

83. The software data structure of claim 82, wherein said confirmed biological function is contribution to a mature mRNA transcript.

84. The software data structure of claim 83, wherein said prediction is output from GenScan.

85. The software data structure of claim 83, wherein said prediction has been confirmed by the method of claim 21.

86. The software data structure of claim 82, wherein said software relating means is the common inclusion of said confirmed prediction data in a single record with said sequence identifier.

87. The software data structure of claim 82, wherein said software relating means links said sequence identifier to confirmed prediction data present in a distinct record.

88. The software data structure of claim 82, wherein said sequence entries further comprise:
software means for relating said sequence identifier to data that encode at least one nucleic acid sequence identified by said identifier.

89. The software data structure of claim 88, wherein said sequence entries further comprise:
software means for relating said sequence identifier and/or said at least one nucleic acid sequence to data that encode a measure of similarity of the at least one nucleic acid sequence to at least one nucleic acid sequence prior-accessioned into a database.

90. The software data structure of claim 89, wherein said sequence entries further comprise:
software means for relating said sequence identifier and/or said at least one nucleic acid sequence to data that encode a textual description of said at least one similar prior-accessioned nucleic acid sequence.

91. The software data structure of claim 82, wherein said sequence entries further comprise:
software means for relating said sequence identifier to data that encode a chromosomal map location of the sequence identified by said sequence identifier.

92. An isolated nucleic acid having exons that have been commonly grouped by the method of claim 56.
